(19) United States
(12) Patent Application Publication
(10) Pub. No.: US 2024/0299737 A1
(43) Pub. Date: Sep. 12, 2024

(54) A STENT-ELECTRODE INTRAVASCULAR NEUROMODULATOR AND ASSOCIATED METHODS FOR ACTIVATION OF A NERVE

(71) Applicant: GALVANI BIOELECTRONICS LIMITED, Brentford, Middlesex (GB)

(72) Inventors: Rizwan BASHIRULLAH, Collegeville, PA (US); Gerald Edwin HUNSBERGER, Collegeville, PA (US)

(73) Assignee: GALVANI BIOELECTRONICS LIMITED, Brentford, Middlesex (GB)

(21) Appl. No.: 18/573,365

(22) PCT Filed: Jun. 23, 2022
(86) PCT No.: PCT/GB2022/051621

§ 371 Date: Dec. 21, 2023

Related U.S. Application Data
(60) Provisional application No. 63/202,766, filed on Jun. 23, 2021.

(51) Int. Cl. A61N 1/05

(52) U.S. Cl. CPC A61N 1/0558

(57) ABSTRACT

Stents for intravascular neural stimulation are disclosed herein that at least partially contact a vessel wall. The stent comprises a scaffold extending in a longitudinal direction and having an outer perimeter that at least partially contacts the wall. A pulse generator can generate electrical signals for delivery to a nerve for intravascular neural stimulation. The scaffold has mounted thereon a first set of one or more electrodes electrically coupled to the pulse generator. The stent further comprises a second set of one or more electrodes electrically coupled to the pulse generator. The second set of electrodes is unconnected to the scaffold, i.e. not directly mounted on the scaffold. Methods of implanting a stent into a vessel using a deployment catheter are disclosed herein. The stent to be implanted comprises a pulse generator, a scaffold, and a distal set of one or more electrodes electrically coupled to the pulse generator. The distal set of electrodes is unconnected to the scaffold, i.e. not directly mounted on the scaffold. The method comprises positioning a distal end of the deployment catheter at the intravascular location, advancing the stent within the deployment catheter, further advancing the stent to expose the one or more electrodes of the distal set of one or more electrodes outside of, preferably beyond the deployment catheter, providing an electrical stimulation via the exposed electrodes, and withdrawing the stent within the deployment catheter.

### FIELD

This disclosure relates to neuromodulation devices and methods for non-destructively stimulating a nerve.

### BACKGROUND

Electrical devices of various shapes and sizes including one or more electrode have been used for neurostimulation of target anatomy. An intravascular device which can effectively stimulate a target nerve is desirable, as such intravascular device may be less invasive than other forms of devices with electrodes.

In embodiments shown in this application, the device takes the form of a stent that is configured to carry electrodes. Placing electrodes, especially of different polarities, on a stent structure presents challenges that are solved by the present invention.

### SUMMARY

In one aspect, the present invention provides a stent for intravascular neural stimulation. The stent comprises a scaffold extending in a longitudinal direction and having an outer perimeter positioned, in use, to at least partially contact the vessel wall. The stent further comprises a pulse generator configured to generate electrical signals for delivery to a nerve for intravascular neural stimulation. The scaffold has mounted (optionally directly mounted) thereon a first set of one or more electrodes electrically coupled to the pulse generator. The stent further comprises a second set of one or more electrodes electrically coupled to the pulse generator. The second set of electrodes is unconnected to the scaffold (i.e. not directly mounted on the scaffold).

At least a part of the first and/or second set of electrodes may be configured to be aligned with at least a part of the outer perimeter of the scaffold such that the set of electrodes are in signalling contact with the vessel wall when the stent is in situ.

The stimulating signal may be a blocking stimulation.

In the case of a stent for providing intravascular neural stimulation, it can be advantageous to mount one or more electrodes somewhere other than the stent scaffold. In such cases, the electrodes can be placed not mounted to the scaffold but in a position such that they will be in signalling contact with the vessel wall when the stent is in situ, and to that end the inventors configured the stent such that these electrodes are configured to be aligned with the outer perimeter of the scaffold.

The scaffold may comprise a proximal end and a distal end. In that case, the first and/or second set of one or more electrodes may be configured such that they extend or be placed distally of (or beyond) the distal end of the scaffold. Certain advantages can follow from placing the electrodes that are not mounted to the scaffold distally from the scaffold rather than at the same longitudinal position or proximally.

The first set of one or more electrodes may be formed at an end or an edge portion of the scaffold such that the electrodes extend or are placed distally of/at the distal end of the scaffold.

The second set of one or more electrodes may be mounted (optionally directly mounted) to the pulse generator. A convenient place to locate the electrodes that are not on the scaffold is the pulse generator. This avoids the need for additional structures.

Optionally, the pulse generator is attached to the scaffold and extends beyond the distal end of the scaffold. In that case, the one or more electrodes are mounted (optionally directly mounted) to the pulse generator such that the one or more electrodes are positioned distally of the distal end of the scaffold.

Optionally, the pulse generator is attached to an inner perimeter of the scaffold. In this way, the electrodes that are mounted (optionally directly mounted) to the pulse generator are in close proximity to or contact with the vessel wall when the stent is in situ.

Optionally, the pulse generator is attached to an outer perimeter of the scaffold. In this way, the electrodes that are mounted (optionally directly mounted) to the pulse generator are in close proximity to or contact with the vessel wall when the stent is in situ. Other means of attachment of the pulse generator to the scaffold are also possible.

In the case where the pulse generator is attached to an outer perimeter of the scaffold, the scaffold may comprise one or more platforms. Each platform may extend around at least part of the circumference of the scaffold, and the pulse generator may be housed on or in one of the one or more platforms. Each platform of the one or more platforms may comprise a surface that extends radially inwardly to form a trough, and the pulse generator may be housed in the trough of the one of the one or more platforms. Optionally the stent may comprise a plurality of platforms or troughs, and a corresponding plurality of pulse generators, each housed in a respective platform or trough. Wherever the term ‘trough’ is used throughout this description a ‘platform’ may be used instead, and vice versa. In that sense, the terms platform and trough may be used interchangeably throughout the description, and should be taken to refer to a space formed on or in the outer perimeter of the scaffold in which a pulse generator can be housed.

In the case of a scaffold comprising one or more platforms or troughs, the pulse generator may be housed on or in such features of the scaffold. In such configurations, the one or more electrodes mounted (optionally directly mounted) on the pulse generator may contact the vessel wall in place of at least part of the outer perimeter of the stent. This is a space-efficient arrangement.

Optionally, the pulse generator and trough in which it is housed may be configured such that the radially outermost part of the pulse generator is aligned with at least a part of the outer perimeter of the scaffold. In such cases, the one or more electrodes mounted (optionally directly mounted) on the pulse generator may be mounted on the radially outermost part of the pulse generator such that, in use, they at least partially contact the vessel wall.

The pulse generator may extend at least partially along the length of the scaffold, or the pulse generator may extend fully along the length of the scaffold. Optionally, the pulse generator may be configured such that it extends distally of (or beyond) the distal end of the platform. The pulse generator may extend distally of (or beyond) the distal end of the scaffold. The one or more electrodes may be mounted (optionally directly mounted) to the pulse generator such that the one or more electrodes are positioned distally of the distal end of the scaffold. Certain advantages, described elsewhere herein, can follow from placing the pulse generator such that it extends distally from the scaffold.

The one or more electrodes of the pulse generator may be comprised of a conductive cap mounted (optionally directly mounted) at the distal end of the pulse generator. Optionally, the cap comprises a rim that at least partially surrounds the distal end of the pulse generator and which is coplanar with at least part of the vessel wall when the stent is in situ.

Optionally, at least a portion of either or both of the of the scaffold and the pulse generator comprises or is formed from an insulating material such that the one or more electrodes mounted (optionally directly mounted) to the pulse generator are electrically isolated from the one or more electrodes mounted (optionally directly mounted) to the scaffold.

The pulse generator may comprise a housing to which the one or more electrodes are mounted (optionally directly mounted) (for example, in the form of a cap). In this case, the housing is optionally formed from an insulating material selected from the group consisting of: a ceramic material and an epoxy. Optionally the scaffold is formed from an insulating material selected from the group consisting of: a ceramic material and an epoxy. Other insulating materials may be used instead.

Optionally the scaffold is formed from a conductive material, and as such may conduct a signal from the pulse generator to the electrodes mounted (optionally directly mounted) on the scaffold. Optionally the conductive material is selected from the group consisting of: stainless steel, Nitinol and Cobolt Alloy. Other conducting materials may be used instead.

Optionally, the first set of one or more electrodes have a first polarity, in use, and wherein the second set of one or more electrodes have a second polarity, in use, wherein the first polarity is different from the second polarity. For example, the first set of one or more electrodes of a first polarity may be anodes and the second set of one or more electrodes of a second polarity may be cathodes. The first and second sets of one or more electrodes may be electrically insulated from one another when the first polarity is different from the second polarity.

Optionally, the first set of one or more electrodes are stimulating electrodes and the second set of one or more electrodes are return electrodes.

Optionally, the second set of one or more electrodes are stimulating electrodes and the first set of one or more electrodes are return electrodes.

The scaffold may be substantially tubular and configured to conform to the walls of the vessel when the stent is in situ. Other configurations of the scaffold are also possible.

In the context of a stent, a scaffold is a well understood structure which is capable of supporting the external wall of the vessel into which the stent is placed. A scaffold may be a continuous or substantially continuous piece of material or a framework of interconnected members. The scaffold may be substantially annular, including ring-shaped, toroidal and/or cylindrical or any other suitable shape that achieves the aforementioned function.

In some embodiments, the stent may comprise a sleeve or a coating of insulating material, wherein the scaffold is attached to or formed on the sleeve of insulating material. The sleeve of insulating material may cover at least a part of both an inner surface and an outer surface of the scaffold structures. In other words, the scaffold structure may be embedded at least partly within the sleeve with only contact surfaces of the electrodes being exposed.

In some embodiments, the scaffold may at least partially be formed of insulating material.

In some embodiments where the scaffold is formed of an electrically conductive material, the scaffold may function as or provide an electrically conductive path between electrodes. An effect of including insulating material on the inner surface of the stent (i.e. between the central portion of the inside of the blood vessel and the electrode) is to promote outward injection of charge rather than inward. This reduces unwanted conduction of signal via the blood, which could lead to short-circuiting, and facilitates a more effective targeting of nerves, which are located outside of the blood vessel. In some embodiments where the scaffold functions as or provides an electrically conductive path, the insulating material may be provided to provide electrical insulation (e.g. to prevent short circuiting) between electrodes or electrical structures of different polarities.

The anodal electrode may also be referred to as a return electrode, and the cathodal electrode may also be referred to as a stimulating electrode.

The cathodal electrode (or the stimulating electrode) may have a surface area of between 0.1 cm² and 0.01 cm², optionally between 0.04 cm² and 0.08 cm², further optionally between 0.05 cm² and 0.075 cm², still further optionally between 0.06 cm² and 0.07 cm², and further optionally 0.067 cm². The surface area may refer to the total surface area of the cathodal electrodes. In some embodiments, the surface area may be between 0.01 cm² and 0.05 cm² and, optionally between 0.015 cm² and 0.04 cm², further optionally between 0.015 cm² and 0.03 cm², and further optionally 0.02 cm².

If a length and a width of the electrode determine the surface area, a thickness of the cathodal electrode may be between 0.1 μm-0.4 mm, optionally between 1 μm-0.1 mm.

The above described structural features may be applied to or be used in conjunction with any implantable device, in particular intravascular or extravascular device. For example, the intravascular device may be a stent comprising one or more stimulating electrode. The stent may further comprise a pulse generator electrically coupled to the stimulating electrode. For example, the intravascular electrode may be a split-stent.

In some embodiments, the stent may comprise a transducer coupled to the pulse generator and configured to receive energy for delivery of power and/or communications to the pulse generator.

An example of a suitable transducer is an antenna configured to receive EM energy such as RF energy, and convert it into DC power. An alternative suitable transducer is an ultrasonic transducer configured to receive electrical energy and convert it into mechanical energy.

The system may further comprise a battery-operated energiser or charger which may be used to wirelessly power the IPG stimulator via the transducer (e.g. antenna) described above. The powering modality between the charger and the implanted device can be near-field, mid-field, RF or ultrasound. Optionally the battery of the energiser is rechargeable with an external near-field charger.

The frequency used when the powering modality is RF may be between 100 kHz-20 MHZ, optionally between 1 MHz-20 MHz, optionally 6.78 MHz, further optionally 13.56 MHz.

The frequency used when the powering modality is ultrasound may be between 100 kHz-5 MHz, optionally between 200 kHz-2 MHz, further optionally between 1-1.2 MHz.

In some embodiments the stent comprises an energy storage circuit or a battery (for example connected to the pulse generator or the transducer) which can be charged. In other embodiments, the stent may be formed without a battery.

In a further aspect, the present invention provides a method for implantation of a stent for intravascular neural stimulation at an intravascular location using a deployment catheter.

The stent to be implanted comprises a pulse generator, a scaffold that is configured to be withdrawn into the deployment catheter and a distal set of one or more electrodes electrically coupled to the pulse generator. The distal set of electrodes is unconnected to the scaffold (i.e. not directly mounted on the scaffold).

The method of implanting the stent comprises positioning a distal end of the deployment catheter at the intravascular location, advancing the stent within the deployment catheter until it approaches the distal end of the deployment catheter, and further advancing the stent to expose the one or more electrodes of the distal set of one or more electrodes outside of, preferably beyond the deployment catheter. The method further comprises providing an electrical stimulation at the intravascular location via the exposed electrodes, and withdrawing the stent within the deployment catheter.

The scaffold of the stent to be implanted may have mounted (optionally directly mounted) thereon a proximal set of one or more electrodes, these electrodes being electrically coupled to the pulse generator. In such cases, the method of implanting the stent may comprise a step of still further advancing the stent to expose at least one distalmost electrode of the proximal set of one or more electrodes mounted (optionally directly mounted) on the scaffold outside of, preferably beyond the deployment catheter. This step permits a distal end of the scaffold to at least partially expand.

The scaffold of the stent to be implanted may be collapsible. Accordingly, the step of withdrawing the stent may cause the collapsible scaffold to collapse.

In some embodiments of the method, an electrical stimulation is provided prior to at least partial, optionally full, deployment of the stent at the intravascular location. This may allow a medical practitioner or user to determine, based on the electrical stimulation, whether the intravascular location is suitable for deployment of the stent. Several different locations may be considered in such a way, and the stent may be deployed at the location considered to be the most suitable. Alternatively, on identification of a suitable intravascular location, the stent may be fully deployed without a consideration of other locations. If no location is considered suitable for implantation the stent may be withdrawn into the deployment catheter, and either the deployment catheter repositioned or removed from the vessel.

The scaffold of the stent to be implanted may comprise a proximal end and a distal end. In that case, the proximal and/or distal set of one or more electrodes may be configured such that they extend distally of the distal end of the scaffold. The step of further advancing the stent may comprise at least partially exposing the proximal and/or distal set of electrodes prior to exposing at least any part of the scaffold. At least partially exposing the electrodes prior to at least any part of the scaffold allows electrical stimulation of the vessel via the exposed electrodes with minimal deployment, potentially no deployment, of the stent. Correspondingly minimal or no expansion of the scaffold will occur. The lesser the extent of expansion, the easier and thus quicker it is to withdraw the stent back into the deployment catheter.

The distal set of one or more electrodes of the stent to be implanted may be mounted (optionally directly mounted) to the pulse generator. This is a convenient place to locate the electrodes that are not on the scaffold, since this avoids the need for additional structures. The step of further advancing the stent may comprise exposing the distal set of electrodes contemporaneously with the pulse generator. Contemporaneous exposure provides control over the precise location at which stimulation is provided to the vessel wall. This is made possible since the circumferential location of the pulse generator on the scaffold is known, and the electrodes used to provide the stimulation are mounted (optionally directly mounted) on the pulse generator. Knowledge of stimulation location prior to stimulation, which is achievable using this embodiment of the present invention, may be advantageous where certain parts of the vessel wall are less suited to stimulation.

The scaffold of the stent to be implanted may comprise one or more platforms. Each platform may extend around at least part of the circumference of the scaffold, and each platform may comprise a surface that extends radially inwardly to form a trough. The pulse generator may be housed in the trough of the one of the one or more platforms. The trough and the pulse generator may be configured such that the radially outermost part of the pulse generator is aligned with at least a part of the outer perimeter of the scaffold. The one or more electrodes may be mounted (optionally directly mounted) on the radially outermost part of the pulse generator. The step of further advancing the stent may comprise exposing the one or more electrodes contemporaneously with the pulse generator.

The pulse generator of the stent to be implanted may extend distally of (or beyond) the distal end of the platform. The one or more electrodes mounted (optionally directly mounted) to the pulse generator may be positioned distally of the distal end of the scaffold. The step of further advancing the stent may comprise at least partially exposing the one or more electrodes, prior to exposing any part of said scaffold. At least partially exposing the electrodes prior to at least any part of the scaffold, has the above mentioned associated advantages.

The scaffold of the stent to be implanted may be substantially tubular and configured to conform to the walls of the vessel. The step of still further advancing the stent may comprise permitting the distal end of the collapsible scaffold to at least partially expand. Such expansion may be in conformity with the vessel wall in which the stent is situated.

In a further aspect, the invention provides a system for delivery of intravascular stimulation comprising a stent according to any one of above paragraphs and a radio frequency (RF) transmitter configured to transmit RF energy which, when received by the RF antenna of the stent, delivers power and/or communications to the pulse generator of the stent.

Thus, the stent may be formed with or without a charge or energy storage device, such as a battery, a capacitor, super capacitor, electrochemical storage device or an inductor. In either case, the stent may be directly powered by an external powering device. Where the stent comprises a charge or energy storage, the stent may be directly powered by an external powering device or the charge or energy storage may be charged by an external powering device.

Depending on the size of the charge or energy storage device, different energy transfer schedule may be used. Whilst two options are described in relation to FIGS. 9 and 10, other arrangements that is a combination of or mid-way between the two options may also be used. For example, in one extreme case, the charge required to deliver an entire therapeutic session may be stored in one or more of the energy storage device(s), whereas in another extreme case the charge required to stimulate a single pulse is supplied by an external energiser in real time. Each of these extreme cases have their relative advantages. In another case, the charge required to deliver a single pulse or multiple pulses (that make up a part of a single therapeutic session) may be stored in one or more of the energy storage device(s), such that the schedule is somewhere between the two extreme cases.

The above described powering modalities and energy transfer schedules may be applied to or be used in conjunction with any implantable device, in particular intravascular or extravascular device. For example, the intravascular device may be a stent comprising one or more stimulating electrode. The stent may further comprise a pulse generator electrically coupled to the stimulating electrode. For example, the intravascular electrode may be a split-stent disclosed in co-pending PCT application PCT/GB2020/053223, which is incorporated by reference herein in its entirety. As another example, the extravascular electrode may be any cuff, spiral or paddle electrode using a wireless powering or charging.

In some embodiments, the scaffold may comprise one or more hooks or projections, each hook or projection being connected at one end to the substantially annular portion and being unconnected at an opposing end to enable attachment of an electrode to the hook or projection.

In some embodiments, the pulse generator is configured to deliver a signal for intravascular stimulation via the anodal and cathodal electrodes for duration of between 60 seconds and 300 seconds, wherein the signal is formed of a train of pulses, and/or wherein: the pulses have a current amplitudes of between 10 mA and 50 mA, optionally between 20 mA and 40 mA; and/or the pulses have a pulse width of between 1 ms to 4 ms, optionally between 2 ms and 3 ms; and/or wherein either: A: the pulses have a frequency of between 5 Hz and 15 Hz, optionally between 8 Hz and 12 Hz, optionally 10 Hz and the pulse train is delivered according to an ON/OFF cycle having a duty cycle of between 10% and 30%, optionally between 15% and 25%, optionally 20%; or B: the pulses have a frequency of between 0.5 Hz and 1.5 Hz, optionally 1 Hz and the pulse train is delivered continuously.

The above described signal may be applied to or be used in conjunction with any intravascular electrode. For example, the intravascular electrode provided may be a stent comprising a stimulating electrode or a split-stent.

### BRIEF DESCRIPTION OF THE FIGURES

Embodiments of the invention will be described, by way of example, with reference to the following drawings, in which:

FIG. 1 is an embodiment of an exemplary stent-electrode for use in an apparatus or method according to the invention;

FIG. 2 is a cross-section of the stent-electrode of FIG. 1;

FIG. 3A shows an exemplary stent-electrode according to the invention in a compressed state attached to a delivery catheter;

FIG. 3B shows the stent-electrode of FIG. 3A in an expanded state;

FIG. 4A shows another exemplary stent-electrode according to the invention in an expanded state;

FIG. 4B shows the stent-electrode of FIG. 4A in a compressed state;

FIG. 5 is a cross-section of an exemplary stent-electrode for use in an apparatus or method according to the invention showing an arrangement of contact electrodes;

FIG. 6 is a cross-section of three further exemplary stent-electrodes for use in an apparatus or method according to the invention showing arrangements of contact electrodes comprising 4, 6 and 8 contacts per anode/cathode;

FIG. 7 is a diagram of an exemplary stent-electrode according to the invention implanted into a patient and in electrical communication with a charger;

FIG. 8 is a diagram of a system for providing power to an exemplary stent-electrode according to the invention;

FIGS. 9 and 10 are time plots showing relationships between current delivered to an exemplary stent-electrode according to the invention and voltage out during setup, stimulation and standby periods;

FIG. 11 is a plot showing eCAP data for an exemplary stent-electrode according to the invention;

FIG. 12 is a communications and control system for use in an apparatus or method according to the invention;

FIGS. 13A-13C show side profiles and corresponding cross-sections of three scaffolds for use in an apparatus or method according to the invention;

FIG. 14 shows the stent-electrode of FIG. 13A having one platform which comprises one trough, said trough housing a pulse generator therein;

FIG. 15 shows an exemplary stent-electrode attached to a deployment catheter for use in a method according to the present invention;

### DETAILED DESCRIPTION

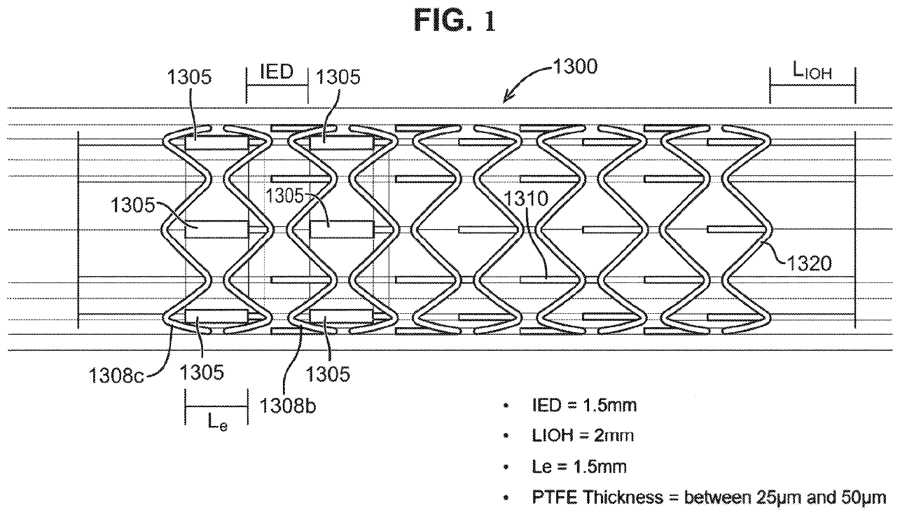

FIG. 1 shows part of an exemplary stent-electrode 1300 (or “stent”) chronic intravascular stimulator according to an aspect of the invention. What is shown in FIG. 1 is a cathode formed of multiple electrodes 1305, and a scaffold 1320 made of 316L stainless steel. The scaffold 1320 is responsible for providing sufficient radial compliance for the stent 1300 to withstand the environment within a blood vessel. Accordingly, the scaffold 1320 provides sufficient, hoop force, mechanical strength and robustness to achieve that function, as well as to allow for crimping and expansion to ensure chronic lifetime survivability. Whilst the scaffold 1320 of the embodiment of FIG. 1 is made from 316L stainless steel, other forms of stainless steel may be used, as could Nitinol, Cobolt Alloy or other stent material optimized for the purpose described.

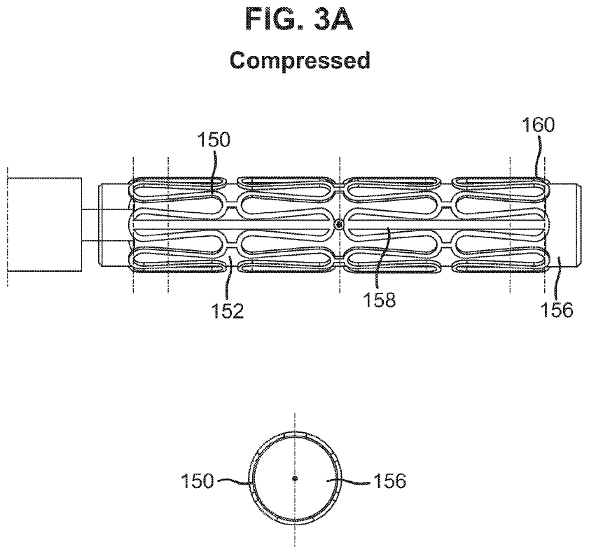

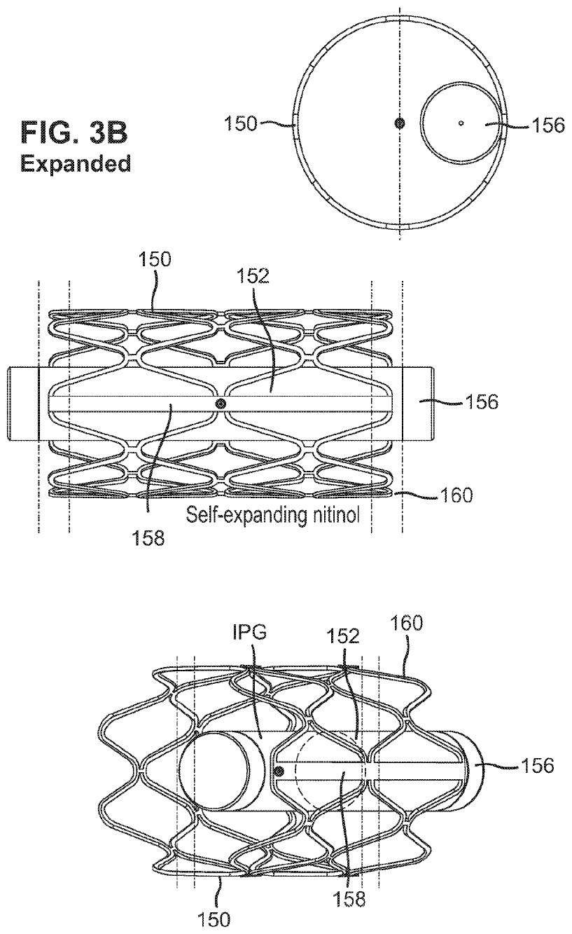

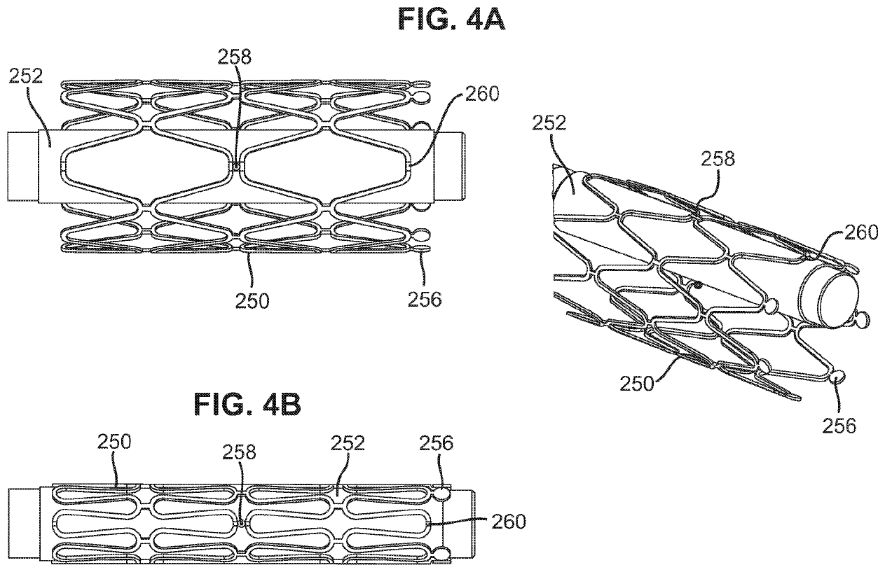

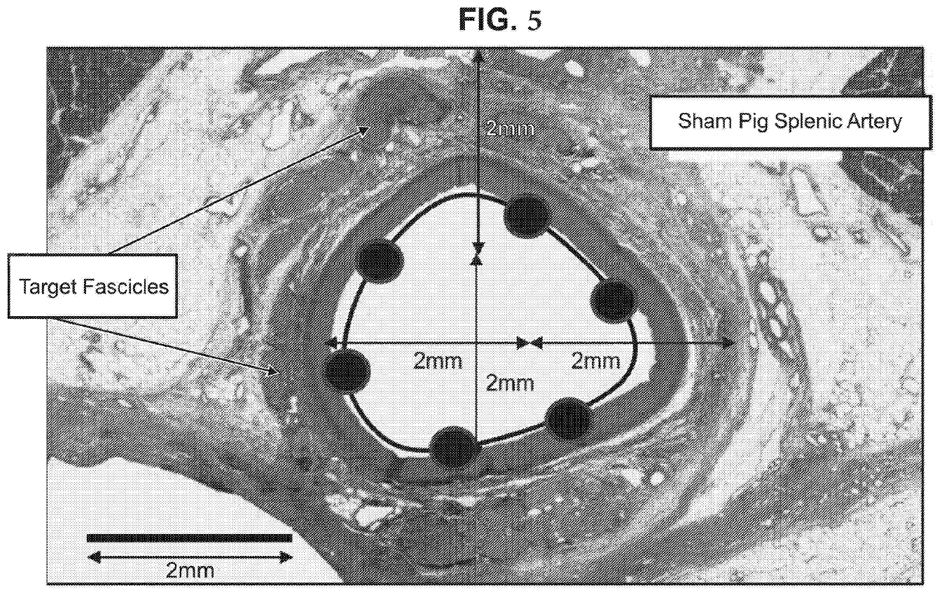

In the embodiment of FIG. 1, electrodes 1305 are cathodes (or “a cathode”), though as explained below, this need not necessarily be the case. What is not shown in FIG. 1 yet forms part of the present invention, as described in more detail below with reference to FIGS. 3 and 4, is an anode and a pulse generator. As described above, the inventors realised that the anode need not be attached to the stent scaffold 1320 in the same way that the cathode is (or, more generally, that electrodes of different polarities need not both be provided on the stent scaffold 1320). Accordingly, in embodiments described elsewhere herein, the anode is mounted to the pulse generator. Of course, that is not the only configuration according to the invention. What is key to the invention is that the anode is configured such that it is aligned with the outer perimeter of the scaffold 1320 such that the electrodes of the anode are in signalling contact with the vessel wall when the stent 1300 is in situ. Though this is conveniently achieved by placing the anode on the pulse generator and attaching the pulse generator to the scaffold 1320 in the manner shown and described elsewhere herein in connection with the embodiment of FIGS. 3 and 4, for example, the same effect can be is achieved with other configurations. It should also be emphasised that whilst the embodiments show a cathode 1305 on the scaffold 1320 of the stent 1300 and an anode elsewhere, the opposite arrangement is also possible whereby an anode is place on the scaffold 1320 and a cathode is placed elsewhere, including on the pulse generator.

Returning to FIG. 1, the cathode 1305 comprises a plurality of contact electrodes 1305 or pads, which may be crimped to the stent scaffold 1320 such that a conductive path is formed between the contact electrodes 1305 and the stent scaffold 1320. Although the contacts may be crimped to the scaffold 1320, other means of attachment could be used, such as welding. For example, the contacts may be laser welded or resistance welded to the scaffold 1320. Other processes for providing mechanical attachment may also be used. In the event that the scaffold 1320 is electrically conductive the attachment process is such that electrical continuity is provided between the contacts 1305 and the stent scaffold 1320. In the event that the scaffold is non-conductive, a separate conductor such as a wire provides electrical continuity between the contacts and the stent scaffold. Alternatively, the contacts could be formed as part of (or all of) the stent scaffold.

The stent-electrode comprises an anode and cathode, though this nomenclature may not always be appropriate in all use-cases for the invention—for instance it will be appreciated that in symmetric biphasic stimulation, each electrode will act as both a cathode and an anode. In this situation, either contact electrode will satisfy the requirements for being an anode and a cathode. In some embodiments, monopolar stimulation may be achieved if an implantable pulse generator is provided, wherein a case or housing of the implantable pulse generator is used as one of the electrode or electrodes. In other embodiments where an implantable pulse generator is provided, the electrode (or a plurality of the electrodes) may be provided (or located) on the implantable pulse generator itself. This is described further in connection with FIGS. 3 and 4 below.

The contact electrodes 1305 of the embodiment of FIG. 1 are made from platinum, though it will be appreciated that other materials may be used in order to achieve the necessary electrical charge injection properties and/or suitability to coating processes that enhance charge storage capacity. For example, the contact electrodes 1305 may comprise platinum, or be formed from an alloy of platinum and iridium, such as an alloy made from 90% platinum and 10% iridium. Alternatively or additionally, the surfaces of the contact electrodes 1305 may be coated possibly with PEDOT, PEDOT:PTS, TiNi, IrOx, PtBlack or treated using a process of laser roughening.

The scaffold 1320 of the stimulator may be attached to a flexible, conforming, insulating material in the form of a sleeve (not shown). The sleeve may be made from high durometer polyurethane, or other materials may be suitable, such as nylon, polyester, or Pebax. The thickness of the polyurethane may be between 25 μm and 50 μm.

The attachment or formation between the scaffold structure 1320 and the sleeve can be achieved in various ways. For example an insulating sleeve may be provided, and the scaffold 1320 may be deposited on the outer surface of the insulating sleeve using a conventional material deposition process. Instead of deposition, the scaffold 1320 may be formed separately and attached to the sleeve by any conventional process such as adhesive or welding. Alternatively, the scaffold 1320 and the sleeve may be manufactured together using, for example, an additive manufacturing process. Alternatively it is possible to manufacture the scaffold structure 1320 and then form the sleeve on the outer surface of the scaffold structure, for example by depositing, overmolding, overlaying or otherwise placing the sleeve on the scaffold structure 1320. In this case, it may be preferable to remove material from the sleeve to reveal parts of the scaffold structure 1320 such as the electrodes 1305. Removing material in this way could be achieved from an etching or laser machining process, for example.

Providing the scaffold structure 1320 is formed from an electrically conductive material, the scaffold structure 1320 may be electrically connected to an implantable pulse generator (discussed elsewhere herein) at a single point, and the electrical path from the implantable pulse generator to each contact electrode 1305 is formed via the scaffold structure 1320. In other words, electrical contact to each exposed electrode 1305 can be achieved by welding or crimping a conductive insulated wire assembly directly onto the stent scaffold 1320 and not to each individual electrodes 1305, thereby minimizing the number of welding points to improve the overall robustness of the stent-electrode design. In other embodiments, a plurality of implantable pulse generators may be provided to electrically connect the scaffold structure 1320 at a single or multiple points.

The arrangement of the electrodes 1305, scaffold structure 1320 and insulating sleeve is such that the anodal and cathodal contact electrodes do not short circuit each other. In one arrangement, a portion of the insulating sleeve may provide separation between the anode and cathode.

In various embodiments of the invention, the insulating material of the sleeve may encapsulate the entirety of the stent scaffold, 1320 leaving only the outwardly facing electrode surfaces exposed towards the endothelial tissue to stimulate the nerves around the artery (or other vessel wall). In such embodiments, it will be appreciated that the total electrode area is determined by the sum of the total exposed electrode material and not the totality of the stent scaffolding material of the corresponding anode or cathode. This allows a degree of freedom and control in the design of the stimulator that allows somewhat independent optimization of its mechanical and electrical functions.

Whilst the scaffold 1320 itself may be conductive, the scaffold may also be non-conductive.

As shown in FIG. 1, in this particular example of a stent-electrode 1300, the inter-electrode distance (IED) is 1.5 mm, the electrode length (Le) is 1.5 mm, the thickness of the PTFE material (see FIG. 14) is between 25 and 50 μm and the length of insulation (in this case the PTFE insulation film) over-hang (L_{IOH}). Of course, it will be appreciated that these dimensions are merely exemplary, and other dimensions will be suitable depending on the particular application. In the example shown, six electrodes 1305 are equally spaced circumferentially around the perimeter of the scaffold 1320 of the stent-electrode 1300, though more or fewer electrodes can be provided, and the spacing can be adjusted depending on the particular application. In the illustrated case, two rings 1308a, 1308b of electrodes 1305 mounted on the scaffold, resulting in a total of twelve electrodes.

In addition, the scaffold 1320 of the stent-electrode 1300 of FIG. 1 is provided with hooks or projections 1310 that, in the illustrated embodiment, extend from the scaffold along the longitudinal axis of the stent at a plurality of locations space apart along the longitudinal axis. These hooks or projections 1310 can be any suitable shape, and are for the purpose of attaching or forming the electrodes 1305. Each hook or projection 1310 of the relevant substantially annular portion 1308a, 1308b carries a respective electrode 1305, though it is not necessary for every hook or projection 1310 of the scaffold 1320 to carry an electrode.

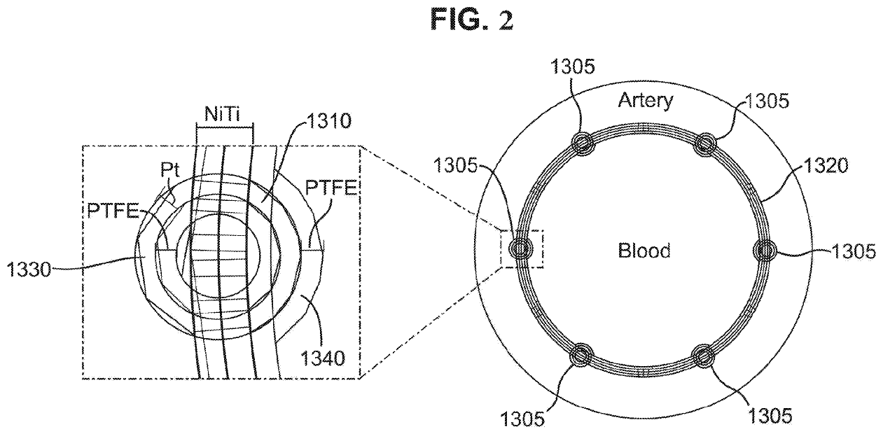

FIG. 2 shows the cross section of the stent-electrode in FIG. 1. The scaffold 1320 of the stent-electrode has a circular cross section, and is made from Nitinol (i.e. an alloy of nickel and titanium). Each hook or projection 1310 has an annular cross-section, though the feature need not be hollow, and could take other cross-sectional shapes. Each hook or projection 1310 may be formed in the same material as the rest of the scaffold or be made from polytetrafluoroethylene (PTFE), though other insulating materials could be used instead. Such insulating materials can be provided at least partly over the hook to provide an increased mechanical attachment between the scaffold hook and the electrode layer or electrode coating, for example the platinum layer 1330. In an embodiment where the scaffold provides an electrically conductive path, at least a part of the electrode layer 1330 is in mechanical and electrical contact with the scaffold hook. In some embodiments, insulating material may not be provided between the scaffold hook 1310 and the platinum layer 1330. Surrounding each hook or projection 1310 is a platinum layer 1330, which forms the electrode 1305 itself. It will be appreciated that in the illustrated embodiment where the stent-electrode scaffold 1320 is conductive, an electrical signal may pass from the scaffold 1320 to the electrode 1305, specifically to the platinum layer 1330. Any suitable electrical connection between these two components will facilitate this electrical coupling. In cases where the scaffold is not conductive, a separate electrical connection, for example a wire, may couple directly to the electrode, for example to a platinum layer similar to the one illustrated.

To prevent an electrical signal passing from the platinum layer toward the inside of the vessel rather than toward the vessel wall as intended, a PTFE coating 1340 is provided over the portion of the platinum layer that is positioned internal to the scaffold 1320; i.e. the portion of the platinum layer that faces radially inwardly. It will be noted that there is no such PTFE coating 1340 provided over the portion of the platinum layer that is positioned external to the scaffold 1320; i.e. the portion of the platinum layer that faces radially outwardly. It will be appreciated that this arrangement promotes outward injection of charge rather than inward, which reduces unwanted conduction of signal via the blood, as described above.

FIGS. 3A and 3B show an exemplary stent for neural stimulation. The stent comprises a scaffold 150, a pulse generator 152, one or more anodal electrodes (not shown) located on the scaffold in a manner similar to that described above in connection with FIGS. 1 and 2, and a cathodal electrode 156 described further below. The stent scaffold may take any shape designed to support the pulse generator 152, to serve as a carrier for the cathode and/or anode electrodes, and to serve as a carrier for any powering/communication coils and/or antenna that may be provided. As mentioned above, in preferred embodiments six electrodes are equally spaced circumferentially around the perimeter of the stent-electrode, though more or fewer electrodes can be provided. Such electrodes may be cathodal or anodal and may be provided in addition to the cathodal electrode 156.

The scaffold 150 is responsible for providing sufficient radial compliance for the stent to withstand the environment within a blood vessel. Accordingly, the scaffold provides sufficient hoop force, mechanical strength and robustness to achieve that function, as well as to allow for crimping and expansion to ensure chronic lifetime survivability. The scaffold may be a mesh structure formed from a wireform pattern of material that takes a substantially tubular-shape overall. A tubular shape is advantageous because the stent conforms to the shape of the vessel in which it is deployed. However, other shapes of scaffold are possible.

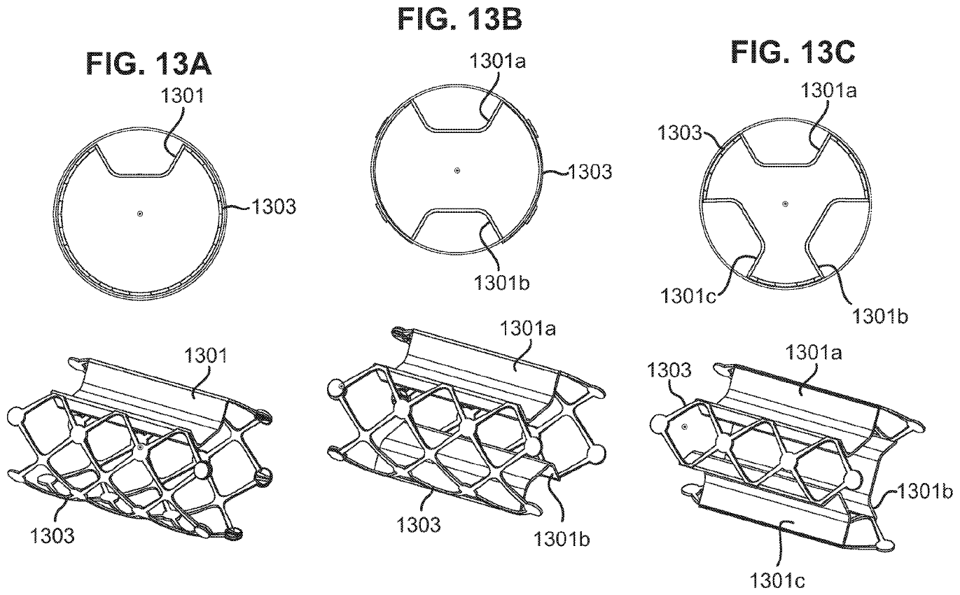

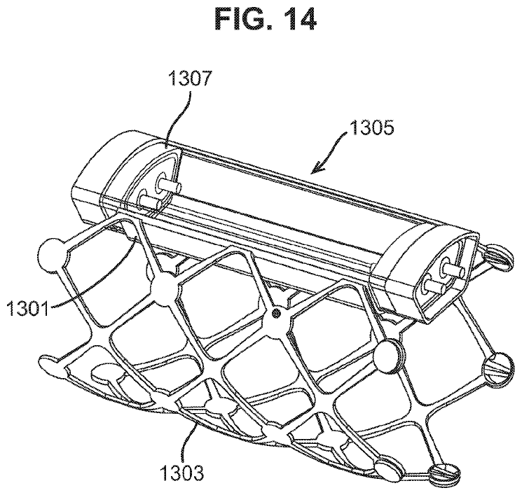

The scaffold may comprise one or more platforms 1301. FIGS. 13A to 13C show three possible configurations for scaffolds, these configurations having one, two and three platforms 1301 respectively. As shown in FIGS. 13A to 13C, each platform 1301a, 1301b and 1301c (collectively, 1301) spans part of the circumference of the scaffold 1303, and each platform 1301 extends along the length of the scaffold 1303. Though FIG. 13 shows the platforms 1301 extending along substantially the full length of their respective scaffold 1303, it is further possible for each platform 1301 to extend along only part of the length of the scaffold 1303 such that scaffold is provided proximally and/or distally of the platform 1301; that is, between the platform 1301 and the proximal and/or distal ends of the stent. Alternatively, it is further possible for each platform 1301 to extend beyond the length of the scaffold 1303, in particular to extent beyond a distal end of the scaffold so as to define the distal end of the stent.

As shown in FIGS. 13B and 13C, where the scaffolds 1303 have two and three platforms 1301, these are spaced equidistantly around the circumference of the scaffold 1303. It will be appreciated that equidistant spacing could be possible for any number of platforms greater than one. However, other configurations, in which the platforms are not spaced equidistance around the circumference of the scaffold, are possible.

As shown in FIGS. 13A to 13C, each platform 1301 comprises a surface that extends radially inward to form a trough. The troughs in the platforms 1301 of FIGS. 13A to 13C comprise a lower (i.e. radially inwardmost) surface that is substantially flat and that extends in a plane that is parallel to the longitudinal axis of the stent and to a tangent of the circumference of the stent. The lower surface is attached to the scaffold 1303 by two obliquely extending side surfaces. This trough configuration provides a stable cradle that is configured to securely house a pulse generator, but alternatives are possible such as a single, continuously curved surface having a concave cross section.

As described below in connection with FIG. 14, each trough is configured to house a pulse generator insofar as the shape of the trough is complementary to the lower (i.e. radially inwardmost) surface of the pulse generator. In other words, the lower surface of the pulse generator may have a shape as described above in connection with the surfaces of the trough so as to match. In embodiments where a plurality of implantable pulse generators are be provided, the scaffold may house one pulse generator in each of the troughs provided in the scaffold. Of course, it may also be possible to house more than one pulse generator in a single trough, such that a scaffold having one platform comprising one trough may house more than one pulse generator. Further, it may be possible to house one of the plurality of implantable pulse generators in a trough, and to mount another of the plurality of implantable pulse generators elsewhere on the scaffold, or elsewhere on the stent device.

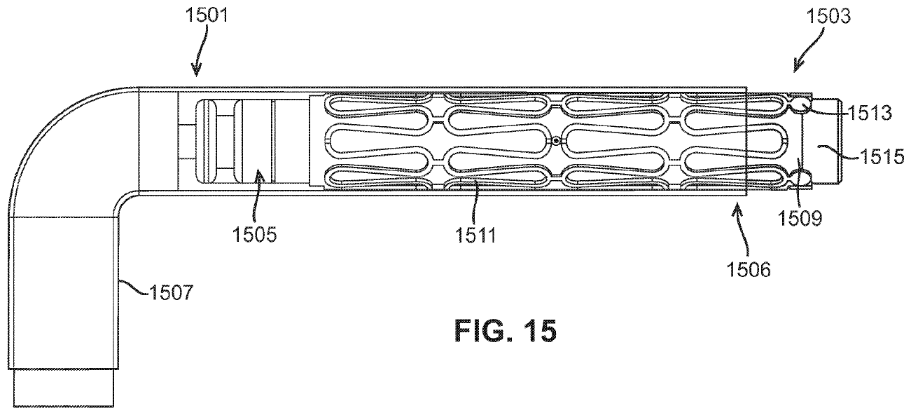

As mentioned above, each platform of those shown in FIGS. 13A to 13C is configured to house a pulse generator. One specific arrangement—in this case of the scaffold 1303 shown in FIG. 13A—is shown in FIG. 14, which shows a pulse generator 1305 housed in the trough of platform 1301 and spanning part of the circumference of the scaffold 1303. As shown, pulse generator 1305 extends beyond—in this case proximally and distally—of the platform 1301. Particular implementations permit the pulse generator to extend proximally and/or distally of the scaffold 1303, for reasons explained in more detail below in connection with FIG. 15.

The attachment between the pulse generator 1305 and scaffold 1303 is described in more detail below. In embodiments where a plurality of implantable pulse generators are be provided, the scaffold may house one pulse generator in each of the platforms provided on said scaffold. As mentioned above, it may also be possible to house more than one pulse generator in a single platform, such that a scaffold having one platform may house more than one pulse generator. Further, it may be possible to house one of the plurality of implantable pulse generators in a platform of the scaffold, and to mount another of the plurality of implantable pulse generators elsewhere on the scaffold, or elsewhere on the stent device.

The scaffold 150 extends in a longitudinal direction, and comprises a proximal end and a distal end 160. According to convention, the proximal end of the scaffold is the end closest to the catheter with which the stent was inserted and the distal end 160 is opposite to that. The proximal end is conventionally upstream (in accordance with the direction of blood flow) in a vessel whereas the distal end 160 is conventionally downstream.

The scaffold 150 has an outer perimeter positioned in use such that the scaffold 150 at least partially contacts the vessel wall. The scaffold may be formed from an insulating material, for example, a ceramic material or epoxy. Alternatively the scaffold may be formed from a conductive material, for example, nitinol or cobalt. It is appreciated however that other insulating or conductive materials optimized for the purpose described may be used. In some embodiments at least a portion of the scaffold 150 may comprise or be formed from either a conductive material, or an insulating material. The material of the scaffold is such that the scaffold may be self-expandable, and collapsible. The scaffold may be laser patterned or stamped from an extruded tube of a desired thickness, although other suitable manufacturing processes may be used.

The pulse generator 152 is configured to generate electrical signals of the kind described below for delivery to a nerve for neural stimulation or block. The pulse generator 152 may be substantially cylindrical shaped, however other shapes may be used. The pulse generator comprises a housing which extends in the longitudinal direction, and has a proximal and a distal end. The housing may be formed from an insulating material, for example, a ceramic material or epoxy. Alternatively, the housing may be formed from a conductive material, for example, Titanium. In some embodiments at least a portion of the pulse generator 150 may comprise or be formed from either a conductive material or an insulating material.

The pulse generator 152 is attached to the scaffold 150. The pulse generator 152 may be attached to an inner perimeter of the scaffold 150, or alternatively the pulse generator may be attached to an outer perimeter of the scaffold, however other attachment positions may be used. Attachment may be achieved by welding, for example, laser welding or resistance welding, however other processes for providing mechanical attachment may also be used. For example, mechanical attachment may be provided using mechanical fasteners such as sutures, hooks or clips. In the event that the scaffold is electrically conductive the attachment process is such that electrical continuity is provided between the pulse generator and the stent scaffold. In the event that the scaffold is non-conductive a separate conductor such as a wire provides electrical continuity between the pulse generator and the stent scaffold.

Where the pulse generator is attached to an outer perimeter of the scaffold, the pulse generator may be attached to the scaffold via a platform such as the platforms described above in connection with FIGS. 13A to 13C, each platform comprising trough. It is to be appreciated that although the following arrangement is described in respect of a platform comprising specific shape of trough, the same may be said for a platforms comprising alternative shapes of trough, or where said platform does not comprise a trough.

FIGS. 13A to 13C and FIG. 14 show an arrangement wherein the pulse generator 1305 and the platform 1301a, 1301b, 1301c (and corresponding trough) in which it is housed and to which it is attached may be configured such that the radially outermost surface 1307 of the pulse generator 1305 is aligned with at least a part of the outer perimeter of the scaffold 1303. As described elsewhere herein, the pulse generator may comprise one or more electrodes mounted on the pulse generator. In the case of FIG. 14, electrodes (not shown) are mounted on the radially outermost surface 1307 of the pulse generator 1305 such that, in use, the one or more electrodes (not shown) at least partially contact the vessel wall when the stent is in situ. By mounting the electrodes on the radially outermost surface 1307, a minimal amount of expansion of the scaffold is required to achieve contact between the electrodes and the vessel wall. However, other configurations of the one or more electrodes mounted on the pulse generator are possible.

The pulse generator 1305 of FIG. 14 extends beyond—in this case proximally and distally—the platform 1301 and thus the scaffold 1303. However, the pulse generator 1305 may only extend partially along the length of the scaffold, or may extend fully along the length of the scaffold. Such arrangements are possible irrespective of whether the pulse generator is attached to an inner perimeter or an outer perimeter of the scaffold. As shown in FIG. 14, the pulse generator 1305 extends distally of a distal end of the platform 1301 and extends distally of a distal end of the scaffold 1303. In alternative arrangements, depending on the relationship between the platform and the scaffold, the pulse generator may extend distally of the distal end of the platform 1301 but not of the scaffold 1303, or may extend distally of the distal end of the scaffold but not of the platform. The same applies to the proximal extension of the pulse generator, which may be independent of the distal extension. The one or more electrodes (not shown) mounted on the pulse generator 1305 may be mounted such that the one or more electrodes are positioned distally of the distal end of the scaffold 1303, the consequence of which is described further below in connection with FIG. 15.

In the embodiment shown in FIG. 3A the scaffold 150 has mounted thereon a plurality of anodal electrodes (not shown). The pulse generator 152 has mounted thereon a single cathodal electrode 156, described in more detail below. Although as shown the electrodes of the scaffold are anodal and the electrode of the pulse generator is cathodal, alternatively the electrodes of the scaffold may be cathodal and the electrode of the pulse generator may be anodal. An example of such an embodiment is shown in FIGS. 4A and 4B. The exemplary stent shown in FIGS. 4A and 4B are similar to that shown in FIGS. 3A and 3B comprising a scaffold 250 extending in a longitudinal direction, and comprising a proximal end and a distal end 260, and a pulse generator 252. However, in the embodiment shown in FIGS. 4A and 4B, the cathodal electrodes (or the stimulating electrodes) 256 are provided on the scaffold 250. The anodal electrode may be provided on the conductive cap where the cathodal electrode 156 was provided in FIGS. 3A and 3B, on other parts of the IPG, or on a separate structure. In the specific embodiment shown in FIGS. 4A and 4B, the cathodal electrode 256 extend beyond a distal end 260 of the scaffold 250. Where similar descriptions apply to both FIGS. 3 and 4, an explicit reference to FIGS. 4A and 4B have been omitted for conciseness.

Further or alternatively, the electrode of the pulse generator may be cathodal and additional cathodal electrodes may be provided on the scaffold. Different combinations of polarities of the electrodes may be used on different parts of the stent.

As described elsewhere herein, six electrode contacts are preferred for the electrodes of the scaffold 150 (in this case, the anodal electrodes), but the number of contacts can vary between 4 and 12 depending of the target vessel size, and in some embodiments even more or even fewer electrodes are possible.

The attachment between the pulse generator 152 and the scaffold 150 is provided by an elongate bar 158 of material that extends along the length of the scaffold 150. As shown, the elongate bar 158 is integrally formed from the same material as the scaffold itself, although it could be manufactured separately and coupled to the scaffold 150 in a subsequent manufacturing step. The elongate bar 158 is attached to the pulse generator 152 by conventional attachment techniques such as welding or screws (not shown). An important advantage follows from the elongate bar 158 forming part of the peripheral wall of the scaffold and the pulse generator 152 being attached to it. In short, since the peripheral wall of the scaffold 150 is configured to be in close proximity to, if not in contact with the vessel wall when the stent is in situ, the specific arrangement of the scaffold 150 and the pulse generator 152, namely whereby the pulse generator is attached to the inner wall of the scaffold 150, means that at least a portion of the pulse generator will also be in close proximity, if not in contact with the vessel wall when the stent is in situ without disrupting the contact between the scaffold 150 and the vessel wall. As such, at least a portion of an electrode mounted to the pulse generator 152 will be in close proximity, if not in contact with the vessel wall when the stent is in situ, simply by virtue of being aligned with the outer perimeter of the scaffold and with no part of the pulse generator being located radially outwardly from the scaffold.

Attachments similar to elongate bar 158 may be provided to the embodiment of FIGS. 4A and 4B, for example attachment portion 258. In other embodiments, smaller attachment portions such as 258 may be applied to the embodiment shown in FIGS. 3A and 3B.

It will be appreciated that though the arrangements of FIGS. 3 and 4 are described in detail herein, they are not the only ways in which an electrode can be aligned to the outer perimeter of the scaffold such that when the stent is in situ the electrode is in close proximity to the vessel wall. For example, one or more arms which do not form part of the pulse generator 152 or the scaffold structure 150 may extend from the pulse generator and/or the scaffold structure so as to position one or more electrodes in line with the outer perimeter of the scaffold such that at least a portion of the one or more electrodes mounted to the pulse generator 152 will be in close proximity, if not in contact with the vessel wall when the stent is in situ. These arms may, for example, be resiliently biased such that the electrodes are held in close proximity, if not in contact with the vessel wall when the stent is in situ. Such arms would not be considered to form part of the scaffold structure, however, since they would not have sufficient radial compliance to withstand the environment within a blood vessel. For example, the arms may lack sufficient hoop force, mechanical strength and robustness to achieve the function achieved by the scaffold structure.

In the embodiment of FIG. 3A, the pulse generator 150 is longer in the axial direction (i.e. along the longitudinal axis) than the scaffold, though this is not required. As a consequence of where the pulse generator 152 is attached relative to the scaffold structure, the distal end of the pulse generator extends beyond the distal end of the scaffold. As such, the electrode mounted to the pulse generator 152 may be positioned distally of the distal end 160 of the scaffold 150. One advantage associated with this arrangement is that the electrode mounted to the pulse generator is kept clear of the electrodes that are mounted to the scaffold 150. However, there are further advantages associated with placing the electrode mounted on the pulse generator beyond the distal end of the scaffold and these will be explained further below.

In the embodiment of FIG. 3B, the electrode mounted to the pulse generator comprises a conductive cap 156. The conductive cap 156 is formed of a single piece of conductive material, and comprises a rim that completely surrounds the perimeter of the pulse generator over which the cap 156 sits. The advantage of this arrangement is that at least a portion of the conductive cap 156 (in this case, the rim) lies adjacent to and coplanar with the vessel wall irrespective of the rotational position of the scaffold 150 in the vessel. In other embodiments of the invention, the one or more electrodes of the pulse generator 152 may be mounted elsewhere on the housing of the pulse generator 152, though care will be needed to avoid interference with the electrodes of the scaffold.

In the embodiment shown in FIGS. 3A and 3B, when the stent is in situ in a vessel and in signalling contact with a vessel wall, a signal may pass between the six (or more) anodal electrodes that are mounted circumferentially around the stent scaffold 150 and the endothelial tissue to stimulate the nerves around the vessel wall. The electrical path is completed by the cathodal electrode formed as part of the conductive cap 156, which is in signaling contact with a portion of the vessel wall as described above.

The contact electrodes may be made from platinum though it will be appreciated that other materials may be used in order to achieve the necessary electrical charge injection properties and/or suitability to coating processes that enhance charge storage capacity. Alternatively or additionally, the surfaces of the contact electrodes may be coated possibly with PEDOT, PEDOT:PTS, TiNi, IrOx, PtBlack or treated using a process of laser roughening.

It will be apparent to the skilled person that for the device to function, it is important for the anodal electrodes and cathodal electrodes to be electrically insulated from each other. As explained elsewhere herein, the scaffold 150 may be made from a conductive material such that it can conduct an electrical signal to the anodal or cathodal electrodes mounted thereto. Likewise, the housing of the signal generator 150 may be made from a conductive material such that it can conduct an electrical signal to the cathodal electrode mounted thereto, such as the conductive cap 156. In that case, it is important that the conductive scaffold 150 and the conductive housing 152 are electrically insulated from each other, such as may be achieved by coating both structures with electrically insulating material such as epoxy, and by ensuring that the attachment between the scaffold 150 and the housing of the pulse generator 152 is such that one is electrically isolated from the other. In such embodiments, insulated conductive wires can pass through the conductive housing to carry a signal from the circuitry inside of the pulse generator 152 housing to the conductive scaffold 150.

Of course, in embodiments wherein at least a portion of either or both of the scaffold 150 and the pulse generator 152 comprises or is formed from an insulating material, the one or more cathodal electrodes mounted to the pulse generator 152 will be electrically isolated from the one or more anodal electrodes mounted to the scaffold 150 by virtue of the insulating material of the structures themselves. For example, either or both of the scaffold and pulse generator housing may be formed from an insulating material, for example, a ceramic material or epoxy. In such cases, in such embodiments, conductive wires, insulated where necessary can carry a signal from the circuitry inside of the pulse generator 152 housing to the electrodes mounted on the scaffold 150 and the housing of the pulse generator 152.

It will be appreciated that the stent may be delivered into the target location of the splenic artery by any known technique, such as by balloon expandable catheter. Alternatively the electrodes can be partially deployed and retracted using a wire pulley mechanism that may comprise of one or two wires controlled proximally by the operator. A second deployment mechanism may be used to anchor the device in the vessel using a NiTi anchor released by retracting a wire holding the anchor in place in its collapsed state. This mechanism allows for a controlled release.

Alternatively or in addition, the scaffold may be self-expanding, for example by being made of a self-expanding Nitinol scaffold, and comprise an anchor, and be delivered to the target location of the splenic artery by a selective release mechanism. Partial deployment of the electrodes may be used to determine the optimal placement of the device intraoperatively.

The embodiment of FIG. 15 shows a steerable and retractable stent delivery system 1501 for implantation of a stent 1503 into a vessel at a target intravascular location. The system 1501 comprises a stent 1503, a deployment catheter 1505 and a steerable sheath 1507. The stent may comprise a pulse generator 1509, a scaffold 1511 configured to be withdrawn into the deployment catheter 1505, and a distal set of one or more electrodes (in this case, a distal electrode 1515) electrically coupled to the pulse generator 1509. As shown in FIG. 15, the distal electrode is unconnected to the scaffold 1511 (i.e. not directly mounted on the scaffold) and is positioned such that it extends distally to the distal end of the scaffold 1511, although other configurations are possible.

The steerable and retractable stent delivery system is designed to allow partial deployment of the stent at an intravascular location. The method by which this is carried out involves a number of steps, as follows. Firstly, the distal end of the deployment catheter 1505 is positioned at the intravascular location. Once in position, the stent 1503 is advanced within the deployment catheter 1505 until it approaches the distal end 1506 of the deployment catheter. At this point, the stent is further advanced to expose the one or more electrodes of the distal set of one or more electrodes (in this case, the distal electrode 1515) outside of, and preferably beyond, the distal end 1506 of the deployment catheter 1505. It will be appreciated that where the distal electrode 1515 is exposed outside of but not beyond the distal end 1506 of the deployment catheter, the exposure takes place only at the distal opening of the deployment catheter 1505.

As shown in FIG. 15, where the distal electrode 1515 is advanced sufficiently to be exposed outside of—and in this case beyond—the distal end 1506 of the deployment catheter, the scaffold 1511 is only minimally advanced beyond the distal end 1506 and thus only minimally deployed. As a result, the scaffold 1511 is at least partially, and in some cases may be fully, contained within the deployment catheter such that only some, or in some cases minimal or even no radial expansion of the scaffold 1511 occurs whilst the distal electrode is nevertheless advanced sufficiently to be activated, as further described below.

Once the distal electrode 1511 is exposed, an electrical stimulation is provided at the intravascular location via the exposed distal electrode 1511. It will be appreciated that with only the distal electrode 1511 exposed, the electrical stimulation provided using only the distal electrode 1511 may be monopolar stimulation. However, this is not essential, and bipolar stimulation using the distal electrode 1511 and additional electrodes 1513 is also possible and is further described below. After providing an electrical stimulation, be it monopolar, bipolar, or some other type of stimulation, the stent may be withdrawn within the deployment catheter, as described further below.

As shown in FIG. 15, the scaffold 1511 has mounted thereon a proximal set of one or more electrodes 1513 electrically coupled to the pulse generator 1509. In this case, and prior to providing an electrical stimulation, the method also comprises the step of still further advancing the stent 1503 to expose at least one electrode, for example the distalmost electrode, of the proximal set of one or more electrodes 1513 mounted on the scaffold. The stent 1503 is further advanced at least (but in some situations only) far enough such that the aforementioned at least one electrode (for example, the distalmost electrode) is exposed outside of, and in this case beyond, the deployment catheter 1505. This step permits a distal end of the scaffold 1511 to at least partially expand. In this resulting configuration an electrical stimulation is provided at the intravascular location via the exposed electrodes, including both the distal electrode 1515 and at least (but in some situations only) the distalmost electrode of the proximal set of one or more electrodes 1513. The electrical stimulation provided in this case may be monopolar if the aforementioned exposed electrodes are configured to apply the same polarity, or bipolar if the aforementioned exposed electrodes are configured to apply opposite polarities. An example of a bipolar application of electrical stimulation would be if the distal electrode 1515 was configured to apply a first polarity, and the proximal set of electrodes were configured to apply a second polarity.

In the embodiments of the present invention in which the pulse generator is attached to an inner perimeter of the scaffold at an attachment point, or to an outer perimeter of the scaffold at an attachment point such as a platform or a trough as described above in connection with FIGS. 13A to 13C or FIG. 14, the at least partial expansion of the distal end of the scaffold takes place in a direction away from the pulse generator at all locations about the perimeter of the scaffold other than at the attachment point.

The above described system and associated method allows stimulation or sensing of the arterial tissue in an intraoperative setting. It will be appreciated that as a consequence of performing the method, a medical practitioner applying the signal via a stent that is only minimally deployed, if at all, is able to test whether the intravascular location of the stent is suitable, based on the resulting signal, without fully deploying the stent. If the practitioner determines that the stent is not in a suitable location the stent may be withdrawn within the deployment catheter. The delivery system allows deployment and activation of the electrodes with minimal or no expansion of the scaffold, thus increasing ease of retraction of the scaffold.

To perform the method described above, the scaffold is retractable and in some embodiments may be collapsible from an expanded to a collapsed configuration. To achieve a collapsed configuration the scaffold reduces in length and diameter in the longitudinal and radial directions respectively from its expanded configuration. The scaffold 1511 shown in FIG. 15 is in its collapsed configuration. According to the method described above, withdrawing a stent having a collapsible scaffold within the deployment catheter causes the collapsible scaffold to collapse.

As described elsewhere herein, the proximal and/or distal set of one or more electrodes (for example, the distal electrode 1515 and/or at least (but in some situations only) the distalmost electrode of the proximal set of one or more electrodes 1513) are configured such that they extend distally of the distal end of the scaffold 1511. In such embodiments, the step of further advancing the stent 1503 comprises at least partially exposing the proximal or distal set of electrodes prior to exposing any part of the scaffold. This is advantageous as it allows electrical stimulation of the vessel via the exposed electrodes with minimal deployment, and in some cases no deployment, of the stent. Correspondingly minimal or no expansion of the scaffold 1511 will occur. The lesser the extent of expansion of the scaffold, the easier and thus quicker it is to withdraw the stent back into the deployment catheter.

As explained elsewhere herein, the distal set of one or more electrodes (for example, the distal electrode 1515) is mounted to the pulse generator 1509. In such embodiments, the step of further advancing the stent 1503 comprises exposing the distal set of electrodes (in this case, the distal electrode 1515) substantially contemporaneously with the pulse generator 1509. Contemporaneous exposure of the distal electrode 1515 and the pulse generator 1509 enables the location at which stimulation is provided to the vessel wall to be carefully controlled. In particular, since the circumferential location of the pulse generator 1509 on the scaffold 1511 is known and may be easily visible during (for example) an endoscopic surgical procedure, and since the electrode(s) used to provide the stimulation are mounted on the pulse generator, the surgeon is afforded improved control over the deployment of at least the distal electrode 1515 compared with stents where electrodes are positioned entirely on the scaffold structure, and/or remote from the pulse generator. It will be appreciated that knowledge of the stimulation location prior to stimulation, which is achievable using the embodiment of FIG. 15 of the present invention among others, is particularly advantageous where certain parts of the vessel wall are less suited to stimulation.

As explained elsewhere herein, the scaffold 1511 may comprise one or more platforms such as platforms 1301 described above in connection with FIGS. 13 and 14. In the case of the embodiment of FIG. 14, for example, wherein the pulse generator 1305 is housed in a trough of the platform 1301, the configuration is such that the radially outermost surface 1307 of the pulse generator is aligned with at least a part of the outer perimeter of the scaffold 1303. As explained above, the one or more electrodes (not shown) mounted on the pulse generator 1305 are mounted on the radially outermost surface 1307 of the pulse generator. In such embodiments, the step of further advancing the stent comprises exposing the one or more electrodes contemporaneously with the pulse generator 1305.

As explained elsewhere herein in connection with the embodiment of FIGS. 13A to 13C and 14, the pulse generator 1305 extends distally of a distal end of the platform 1301. As explained above, one or more electrodes (not shown) are mounted to the pulse generator 1305 such that the one or more electrodes are positioned distally of the distal end of the scaffold 1303. In such embodiments, the step of further advancing the stent comprises at least partially exposing the one or more electrodes, prior to exposing at least any part of said scaffold 1303.

Returning to FIG. 15 and other figures showing embodiments in which the one or more electrodes (in the case of FIG. 15, the distal electrode 1515) of the pulse generator comprise a conductive cap mounted at the distal end of the pulse generator 1509, the step of further advancing the stent 1503 comprises exposing the conductive cap such that it is in signalling contact with the vessel wall.

It will be appreciated that in all illustrated embodiments described elsewhere herein, the scaffold of the stent may be substantially tubular and configured to conform to the walls of the vessel in use. In such embodiments the step of still further advancing the stent may comprise permitting the distal end of the scaffold to at least partially expand in conformity with the vessel wall in which the stent is situated. This causes not only the distal set of one or more electrodes (such as distal electrode 1515 mounted on the pulse generator) but also the proximal set of one or more electrodes mounted on the scaffold into contact with the vessel wall, irrespective of the tortuosity of the vessel wall. This ensures that sufficient stimulation of the vessel wall may occur, irrespective of the complexity of the vasculature.

Finally, in the system shown in FIG. 15, it will be appreciated that whilst we have described above a partial deployment followed by retraction, full deployment of the stent is achieved by pushing the deployment catheter in a distal direction whilst pulling the sheath in a proximal direction. Retraction of the stent, irrespective of the extent of its deployment, is achieved by pulling the deployment catheter in the proximal direction whilst pushing the sheath in a distal direction. The present invention is however not limited to this, and other suitable methods of deployment may be used.

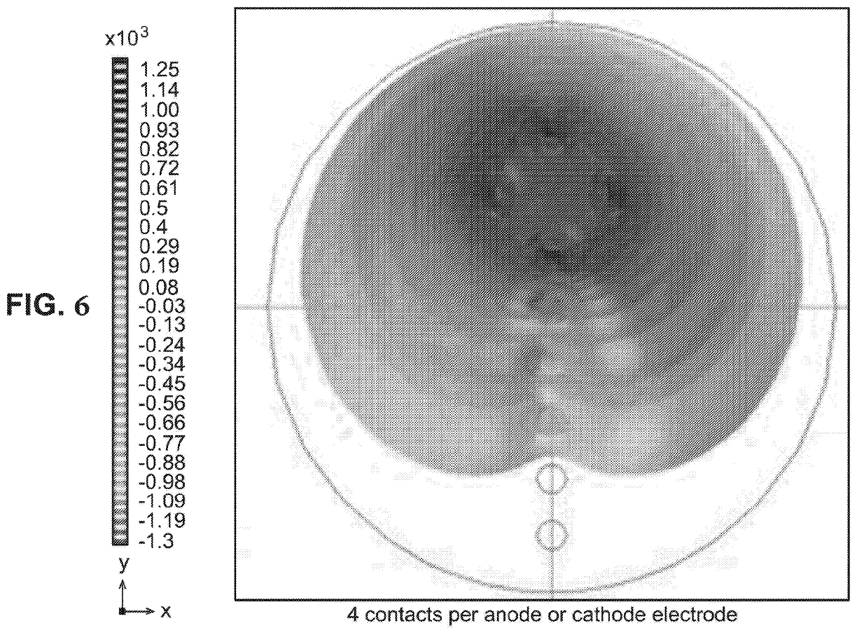

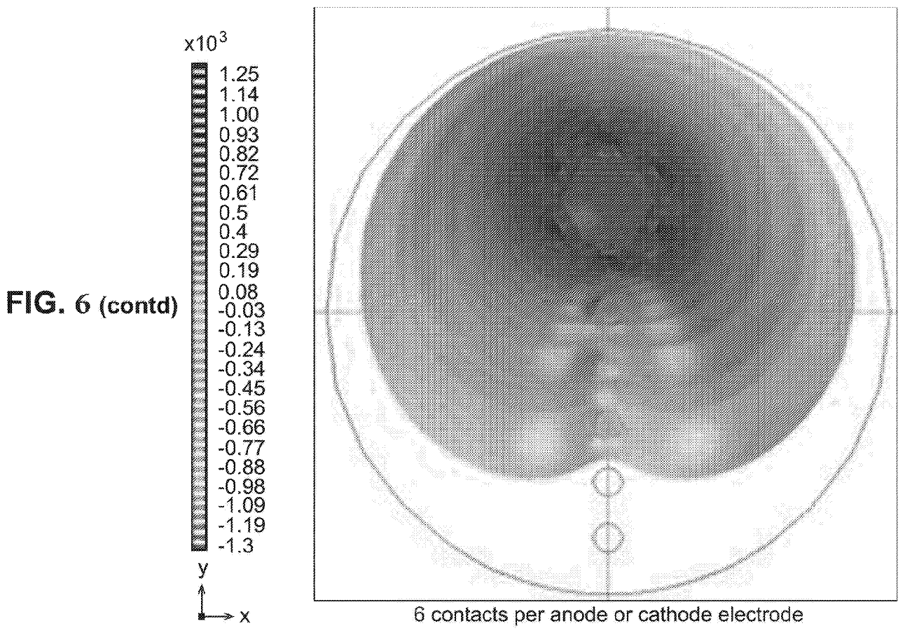

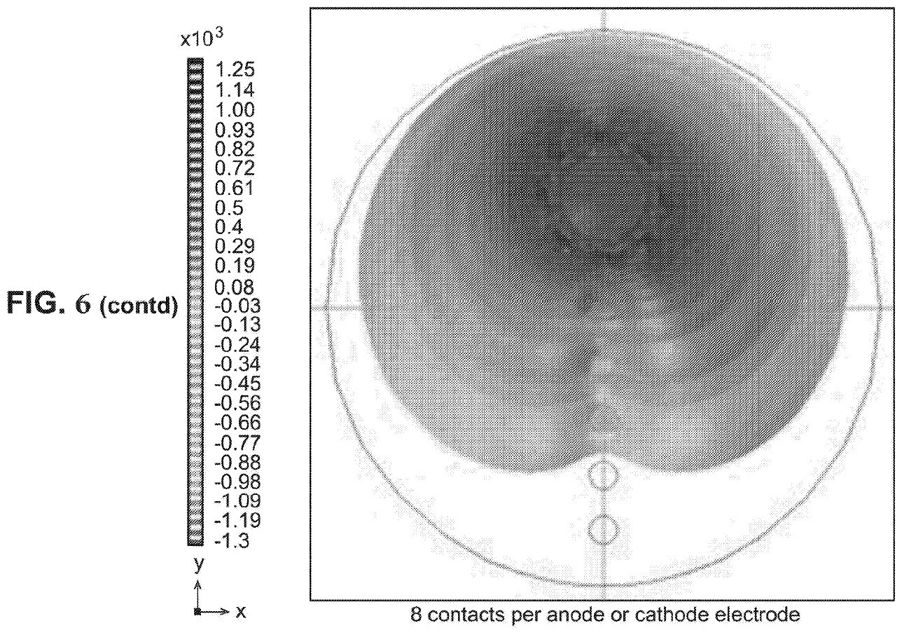

The stent-electrode chronic intravascular stimulators described above comprise multiple electrodes on at least the contacts provided on the stent scaffold, which is typically the anode. In FIG. 2, six electrode contacts per anode are shown, but the number of contacts can vary between 4 and 12 depending of the target vessel size. FIG. 6 shows alternative embodiments where 4, 6 and 8 electrodes are used. Optionally, 4, 6, 8, 10 or 12 electrode contacts per anode are used, but 5, 7, 9 or 11 electrode contacts per anode may be used instead.

Embodiments according to the invention may be implemented using a symmetric or an asymmetric scaffolding configuration. A symmetric design is typically more compact, but asymmetry may be helpful to facilitate placement and to robustly anchor the device onto a target site. Symmetric and asymmetric embodiments may exist with a unitary body. The symmetric scaffolding configuration may exist as an asymmetric embodiment, and the asymmetric scaffolding configuration may exist as a symmetric embodiment.

The stent-electrode chronic intravascular stimulator may comprise a miniature implantable pulse generator (IPG) with wireless antenna for receiving power and communication from a transmitter (described elsewhere herein).

Each contact electrode is coupled to a miniature IPG in any manner as described elsewhere herein.

The antenna may be made of electrically insulated multi-conductor weaved in the stent scaffold and attached to the IPG through hermetic feedthrus. The antenna may be encapsulated within the IPG stimulator itself. In an alternative arrangement the antenna is made of electrically insulated multi-conductor weaved with an anchor (described below). The embodiment in which antenna is encapsulated within the IPG stimulator itself may use ultrasound as the powering modality in addition to, or alternative to those that require an antenna (such as RF). For example, the IPG stimulator may comprise a transducer for delivering electrical energy to the electrodes using energy source such as ultrasound. In other words, some embodiments do not require an antenna.

In a further embodiment the antenna is embedded with the IPG stimulator and anchoring to the artery is accomplished using the NiTi stent scaffold itself. In this case, the scaffold can be made of an expandable insulative polymer matrix. This embodiment may use ultrasound as the powering modality in addition to, or alternative to those that require an antenna (such as RF). For example, the IPG stimulator may comprise a transducer for delivering electrical energy to the electrodes using energy source such as ultrasound.

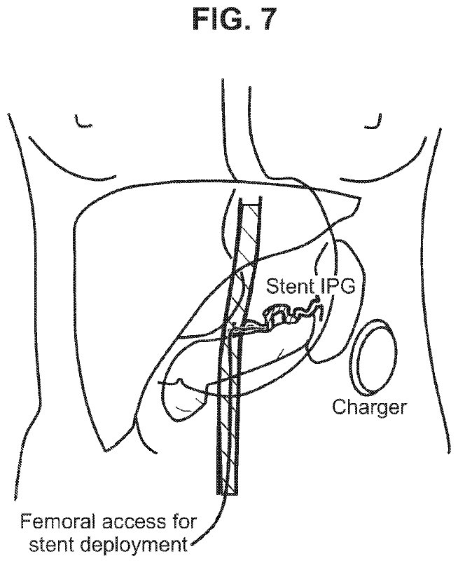

FIG. 7 illustrates a stent as described elsewhere herein as part of a system according to an aspect of the invention. The system comprises the stent-electrode stimulator, which is shown in FIG. 7 as deployed into the splenic artery through femoral access using a 7Fr to 9Fr delivery system. The system further comprises battery-operated energizer or charger which is used to wirelessly power the IPG stimulator via the antenna described above. The powering modality between the charger and the stent-electrode IPG stimulator can be near-field, mid-field or ultrasound. Optionally the battery of the energizer is rechargeable with an external near-field charger.

The energizer (and optionally its charger) may be a wearable device, or may be implanted in a subcutaneous pocket of a patient. A wearable device may be advantageous for ad-hoc stimulation and/or where the charger requires frequent recharging. Conversely, an implantable device may be advantageous to deliver continuous stimulation, or deliver a scheduled therapy on a program, wherein the powering modality between the implanted charger and the IPG stimulator can be near-field, mid-field or ultrasound.

FIG. 8 shows a power system for the devices described in connection with FIG. 7. As described in more detail below, energy supplied by the charger is stored and accumulated within the IPG in storage elements such as super-capacitors, and subsequently used to apply therapy.

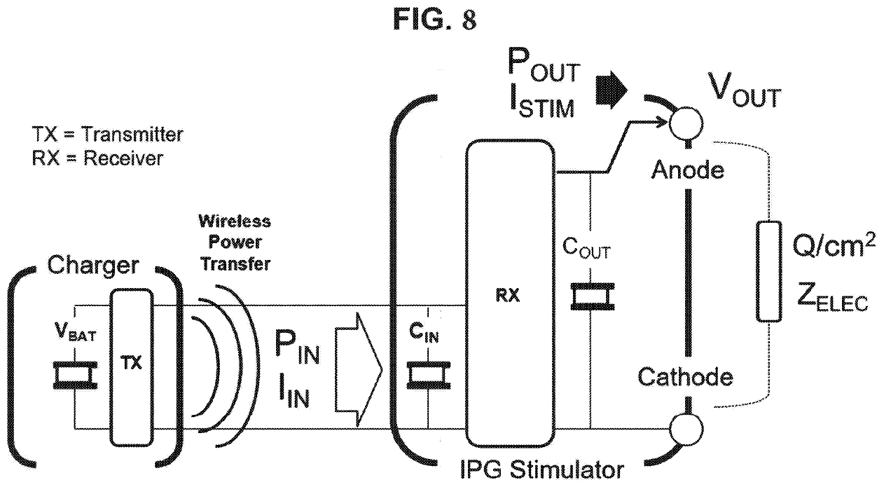

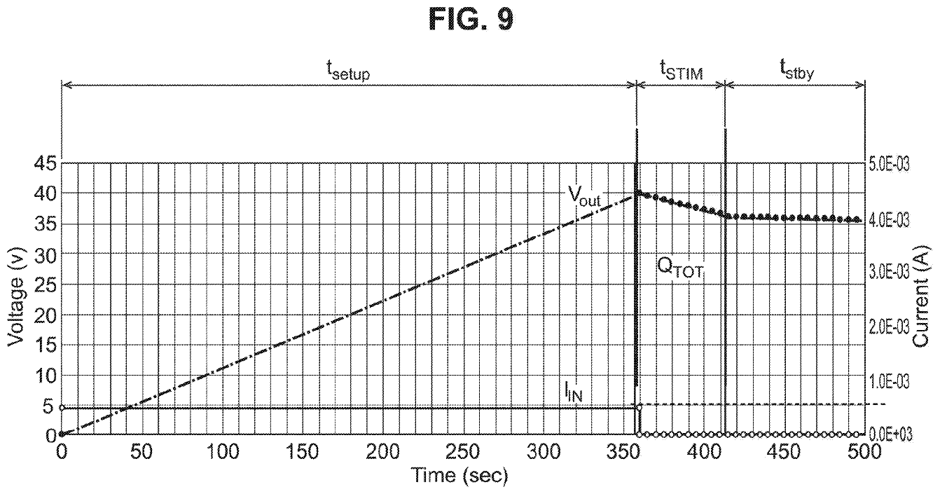

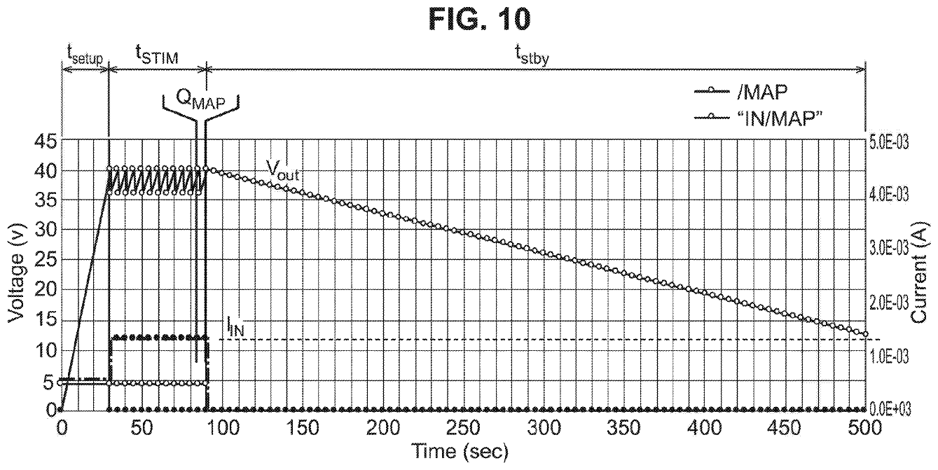

FIG. 9 shows an energy transfer schedule applicable to the system of FIGS. 7 and 8. In this case, energy supplied from a wireless charger is stored in capacitors until the total therapeutic dose is accumulated, at which time it is used to apply therapy. Compared with the system of FIG. 10, this system requires larger capacitors and a longer time to accumulate the charge for stimulation. On the other hand this system requires a lower input energy to ‘trickle-charge’ the capacitor or other storage elements to the desired output voltage level. Moreover, it only requires energy to be supplied continuously until the required voltage is reached, at which point the charger can cease delivery of energy.

FIG. 10 shows an alternative energy transfer schedule applicable to the system of FIGS. 7 and 8. Again, in this case energy supplied from a wireless charger is stored in capacitors until the charge required to deliver a micro-burst is accumulated. In particular, this is the charge required to deliver an ‘active period’ dose comprising of a burst of pulses in one active period. Once the required charge is accumulated, it is used to apply therapy. Compared with the system of FIG. 9, this system requires smaller capacitors, and less time to accumulate the charge for stimulation. On the other hand, the system requires slightly higher input energy to charge the capacitor or other storage elements to the desired output voltage level. Moreover, it requires energy supplied continuously through the duration of the therapy.

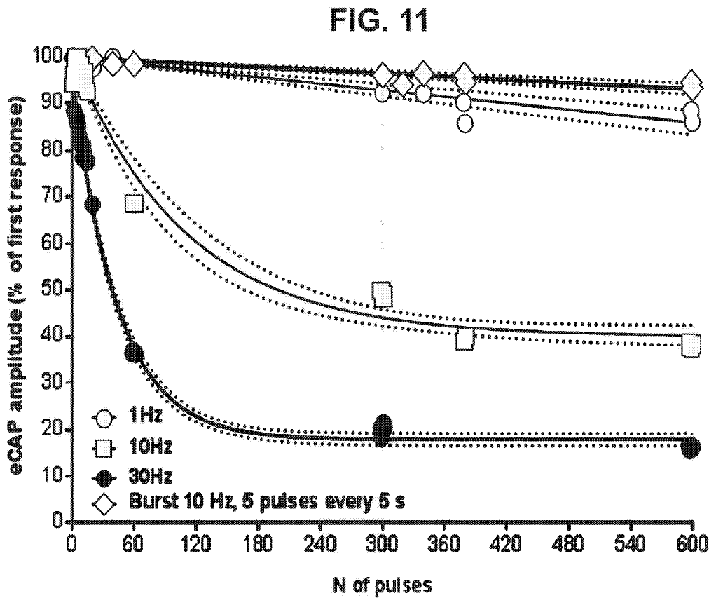

FIG. 11 shows eCAP amplitude (in percentage of first responses) over number N of pulses for different signal parameters, specifically 1 Hz. 10 Hz, 30 Hz continuous, and a burst pattern of 10 Hz comprising 5 pulses every 5 seconds. In one embodiment, preferred parameters of stent-electrode stimulation systems described elsewhere herein are 10 Hz pulses with 0.5 sec ON time and 5 sec OFF time at current amplitudes ranging from 10 mA to 40 mA and pulse widths ranging from 1 ms to 4 ms, for a total duration of 60 sec up to 300 sec. In another embodiment, preferred parameters of stent-electrode stimulation systems described elsewhere herein are 1 Hz pulses delivered continuously at current amplitudes ranging from 10 mA to 40 mA and pulse widths ranging from 1 ms to 4 ms, for a total duration of 60 sec up to 300 sec. This will achieve the same therapeutic effect as 10 Hz, 0.5 sec/5 sec ON/OFF parameters but with less peak input energy demands.

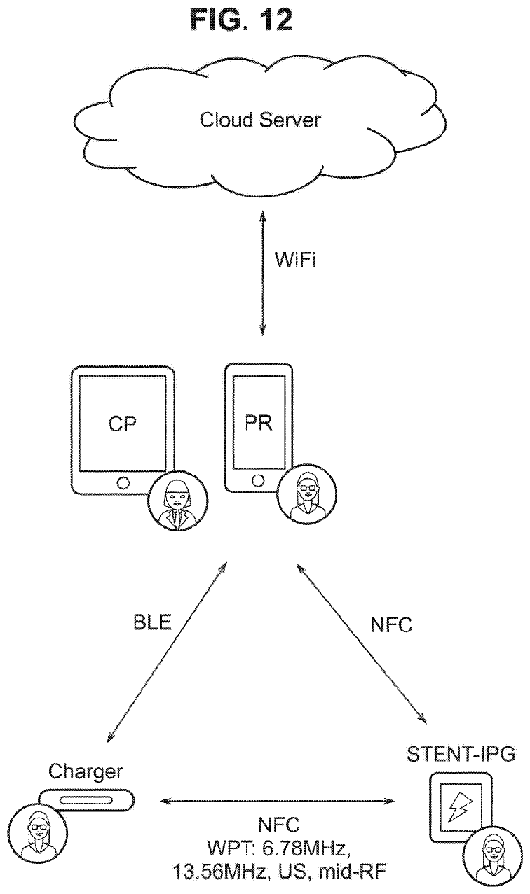

FIG. 12 shows a communications and control system according to the invention. The system comprises the stent-IPG stimulator and charger described elsewhere herein, and a deployment catheter. It also comprises a patient remote (PR) and clinician programmer (CP).

In a preferred embodiment, the CP connects to the charger via BLE to program therapy parameters in non-volatile memory. Therapy parameters may also be programmed into the stent IPG non-volatile memory. The CP & PR are used to monitor therapy by communicatively coupling with the charger while the charger energizes the IPG stent implant to deliver therapy.

In the illustrated embodiment, the CP/PR software applications serve as the gateway to cloud connectivity and are used to download therapy parameters, track compliance and send patient reminders. Additionally the CP/PR can connect directly to the stent implant via NFC link for diagnostics or monitoring purposes.

In the illustrated embodiment, the charger energizes the stent-IPG through wireless powering scheme based on 6.78 MHz or 13.56 MHz ISM bands, ultrasound or mid-field powering. It supports BLE with the CP/PR and NFC with the stent IPG. It will be appreciated that other wireless powering schemes may be used, and other communication protocols may be used for communication.

In some embodiments, the stent-IPG stimulator is energized by the charger while therapy is being delivered. It may also link to external devices such as the charger and CP/PR over the NFC protocol. The stent-IPG is a single-fault safe device since it does not contain a battery and is not intended to be explanted in the event of failure.

In summary, the charger of the system of FIG. 12 energizes the stent-IPG stimulator via NFC to deliver therapy and links to the CP/PR via BLE. The CP/PR of FIG. 12 provides patient app gateway to the cloud in order to download/upload therapy parameters, track compliance and sends reminders. The CP/PR also links to the charger via BLE to monitor progress during therapy and links to the stent-IPG stimulator via NFC for diagnostics and monitoring. Finally, the stent-IPG stimulator is energized by charger while therapy is delivered and links to Charger or CP/PR via NFC. It is a single-fault safe device, by which it is meant that it does not comprise a battery.

The stent may be positioned inside the splenic artery for stimulating the splenic nerve or branches of the splenic nerve.

The stent discussed in this application could be used in conjunction with any suitable blood vessel in order to apply an electrical signal to any corresponding nerve. Other examples include: the carotid artery and the vagus nerve and the cervical sympathetic ganglion; the aorta and the phrenic nerve, the vagus nerve, the superior mesenteric ganglion, and the inferior mesenteric ganglion; the renal artery and the renal nerves; and the subclavian artery and the brachial plexus; and common hepatic artery and its associated nerves; and gastroduodenal artery and its associated nerves; iliac artery and splanchnic nerves.

The invention may be useful for treating subjects who are suffering from, or who are at risk of developing, diseases, disorders or conditions associated with inflammation, e.g. inflammatory disorders, e.g., autoimmune disorders. The invention may treat or ameliorate the effects of such diseases, disorders or conditions by reducing inflammation. This may be achieved by decreasing the production and release of pro-inflammatory cytokines, and/or by increasing the production and release of anti-inflammatory cytokines and pro-resolving molecules, from the spleen, by electrically stimulating the splenic arterial nerve as described herein.

Inflammatory disorders include autoimmune disorders, such as arthritis (e.g. rheumatoid arthritis, osteoarthritis, psoriatic arthritis), Grave's disease, myasthenia gravis, thryoiditis, systemic lupus erythematosus, Goodpasture's syndrome, Behcets's syndrome, allograft rejection, graft-versus-host disease, ankylosing spondylitis, Berger's disease, diabetes including Type I diabetes, Reitier's syndrome, spondyloarthropathy psoriasis, multiple sclerosis, Inflammatory Bowel Disease, Crohn's disease, Addison's disease, autoimmune mediated hair loss (e.g., alopecia areata) and ulcerative colitis.

Certain examples of inflammatory disorders include diseases involving the gastrointestinal tract and associated tissues, such as appendicitis, peptic, gastric and duodenal ulcers, peritonitis, pancreatitis, ulcerative, pseudomembranous, acute and ischemic colitis, inflammatory bowel disease, diverticulitis, cholangitis, cholecystitis, Crohn's disease, Whipple's disease, hepatitis, abdominal obstruction, volvulus, post-operative ileus, ileus, celiac disease, periodontal disease, pernicious anemia, amebiasis and enteritis.

Examples of inflammatory disease, disorders or conditions affecting the bones, joints, muscles and connective tissues include the various arthritides and arthralgias, osteomyelitis, gout, periodontal disease, rheumatoid arthritis, spondyloarthropathy, ankylosing spondylitis and synovitis.

Further examples include systemic or local inflammatory diseases and conditions, such as asthma, allergy, anaphylactic shock, immune complex disease, sepsis, septicemia, endotoxic shock, eosinophilic granuloma, granulomatosis, organ ischemia, reperfusion injury, organ necrosis, hay fever, cachexia, hyperexia, septic abortion, HIV infection, herpes infection, organ transplant rejection, disseminated bacteremia, Dengue fever, malaria and sarcoidosis.

Other examples include diseases involving the urogential system and associated tissues, such as diseases that include epididymitis, vaginitis, orchitis, urinary tract infection, kidney stone, prostatitis, urethritis, pelvic inflammatory bowel disease, contrast induced nephropathy, reperfusion kidney injury, acute kidney injury, infected kidney stone, herpes infection, and candidiasis.

Other examples include involving the respiratory system and associated tissues, such as bronchitis, asthma, hay fever, ventilator associated lung injury, cystic fibrosis, adult respiratory distress syndrome, pneumonitis, alvealitis, epiglottitis, rhinitis, achlasia, respiratory syncytial virus, pharyngitis, sinusitis, pneumonitis, alvealitis, influenza, pulmonary embolism, hyatid cysts and/or bronchiolitis.

Further examples are dermatological diseases and conditions of the skin (such as burns, dermatitis, dermatomyositis, burns, cellulitis, abscess, contact dermatitis, dermatomyositis, warts, wheal, sunburn, urticaria warts, and wheals); diseases involving the cardiovascular system and associated tissues, (such as myocardial infarction, cardiac tamponade, vasulitis, aortic dissection, coronary artery disease, peripheral vascular disease, aortic abdominal aneurysm, angiitis, endocarditis, arteritis, atherosclerosis, thrombophlebitis, pericarditis, myocarditis, myocardial ischemia, congestive heart failure, periarteritis nodosa, and rheumatic fever, filariasis thrombophlebitis, deep vein thrombosis); as well as various cancers, tumors and proliferative disorders (such as Hodgkin's disease), nosocomial infection; and, in any case the inflammatory or immune host response to any primary disease.

Other examples of inflammatory disorders include diseases involving the central or peripheral nervous system and associated tissues, such as Alzheimer's disease, depression, multiple sclerosis, cerebral infarction, cerebral embolism, carotid artery disease, concussion, subdural hematoma, epidural hematoma, transient ischemic attack, temporal arteritis, spinal cord injury without radiological finding (SCIWORA), cord compression, meningitis, encephalitis, cardiac arrest, Guillain-Barre, spinal cord injury, cerebral venous thrombosis and paralysis.

Inflammatory disorders also include conditions associated with immune or inflammatory response (i.e. acute inflammatory episodes) include injury to nerves or other tissue and pain associated with nerve or other tissue. Injury may be due to a physical, chemical or mechanical trauma. Non-limiting examples of injury include acute trauma, burn, whiplash, musculoskeletal strains, and post-operative surgery complications, such as DVT, cardiac dysrhythmia, ventilator associated lung injury, and post-operative ileus.

Conditions associated with a particular organ such as eye or ear may also include an immune or inflammatory response such as conjunctivitis, iritis, glaucoma, episcleritis, acute retinal occlusion, rupture globe, otitis media, otitis externa, uveitis and Meniere's disease. Another example of an inflammatory disorder is post-operative ileus (POI). POI is experienced by the vast majority of patients undergoing abdominal surgery. POI is characterized by transient impairment of gastro-intestinal (GI) function along the GI tract as well pain and discomfort to the patient and increased hospitalization costs.

The impairment of GI function is not limited to the site of surgery, for example, patients undergoing laparotomy can experience colonic or ruminal dysfunction. POI is at least in part mediated by enhanced levels of pro-inflammatory cytokines and infiltration of leukocytes at the surgical site. Neural inhibitory pathways activated in response to inflammation contribute to the paralysis of secondary GI organs distal to the site of surgery. Stimulation of neural activity as taught herein may thus be effective in the treatment or prevention of POI.

The invention is particularly useful in treating autoimmune disorders (e.g. rheumatoid arthritis, osteoarthritis, psoriatic arthritis, spondyloarthropathy, ankylosing spondylitis, psoriasis, systemic lupus erythematosus (SLE), multiple sclerosis, Inflammatory Bowel Disease. Crohn's disease, and ulcerative colitis) and sepsis.

This invention is particularly useful for treating B cell mediated autoimmune disorders (e.g. systemic lupus erythematosus (SLE) and rheumatoid arthritis (RA)).

The invention is particularly useful for treating inflammatory conditions associated with bacterial infections. For example, the invention is particularly useful for treating inflammatory conditions caused or exacerbated by Escherichia coli, Staphylococcus aureus, Pneumococcus, Haemophilus influenza, Neisseria meningitides, Streptococcus pneumonia, Methicillin-resistant Staphylococcus aureus (MRSA), Klebsiella or Enterobacter infection. Treatment of the inflammatory disorder can be assessed in various ways, but typically involves determining an improvement in one or more physiological parameters of the subject.

Useful physiological parameters may be one or more of the group consisting of: the level of a pro-inflammatory cytokine, the level of an anti-inflammatory cytokine, the level of a catecholamine, the level of an immune cell population, the level of an immune cell surface co-stimulatory molecule, the level of a factor involved in the inflammation cascade, the level of an immune response mediator, and the rate of splenic blood flow.

Improvement in a determined physiological parameter in the context of the invention may be one or more of the group consisting of: a reduction in a pro-inflammatory cytokine, an increase in an anti-inflammatory cytokine, an increase in a catecholamine, a change in an immune cell population, a change in an immune cell surface co-stimulatory molecule, a reduction in a factor involved in the inflammation cascade, a change in the level of an immune response mediator and a decrease in splenic blood flow. The invention might not lead to a change in all of these parameters.

By stimulating a splenic arterial nerve at a site where the splenic artery is not in direct contact with the pancreas, the spleen may: (a) decrease the secretion of a pro-inflammatory cytokine compared to baseline secretion; and/or (b) increase the secretion of an anti-inflammatory cytokine compared to baseline secretion. For example, the decrease in a pro-inflammatory cytokine secretion may be by: ≤5%, ≤10%, ≤15%, ≤20%, ≤25%, ≤30%, ≤35%, ≤40%, ≤45%, ≤50%, ≤60%, ≤70%, ≤80%, ≤90% or ≤95%. The increase in an anti-inflammatory cytokine secretion may be by: ≤5%, ≤10%, ≤15%, ≤20%, ≤25%, ≤30%, ≤35%, ≤40%, ≤45%, ≤50%, ≤60%, ≤70%, ≤80%, ≤90%, ≤95%, ≤100%, ≤150% or ≤200%.

Once the cytokine is secreted into the circulation, its concentration in the circulation is diluted. Stimulation of the splenic arterial nerve may result in: (a) a decrease in the level of a pro-inflammatory cytokine in the plasma or serum by ≤5%, ≤10%, ≤15%, ≤20%, ≤25%, ≤30%, ≤35%, ≤40%, ≤45%, ≤50%, ≤60%, ≤70%, ≤80%, ≤90%, or ≤95%; and/or (b) an increase in the level of an anti-inflammatory cytokine in the plasma or serum by ≤5%, ≤ 10%, ≤15%, ≤20%, ≤25%, ≤30%, ≤35%, ≤40%, ≤45%, ≤50%, ≤60%, ≤70%, ≤80%, ≤ 90%, ≤95%, ≤100%, ≤150% or ≤200%. Preferably the level in the serum is measured. By stimulating the splenic arterial nerve, the level of catecholamine (e.g. norepinephrine or epinephrine), e.g. its level in the spleen, may increase, for example, by: ≤5%, ≤10%, ≤ 15%, ≤20%, ≤25%, ≤30%, ≤35%, ≤40%, ≤45%, ≤50%, ≤60%, ≤70%, ≤80%, ≤90%, ≤ 95%, ≤100%, ≤150% or ≤200%.

For example, the inventors found that stimulating a splenic arterial nerve can decrease the level of a pro-inflammatory cytokine (e.g. TNFα) in the serum by 30%-60%.

Pro-inflammatory cytokines are known in the art. Examples of these include tumor necrosis factor (TNF; also known as TNFα or cachectin), interleukin (IL)-1α, IL-1β, IL-2; IL-5, IL-6, IL-8, IL-15, IL 18, interferon γ (IFN-γ); platelet-activating factor (PAF), thromboxane; soluble adhesion molecules; vasoactive neuropeptides; phospholipase A2; plasminogen activator inhibitor (PA1-1); free radical generation; neopterin; CD14; prostacyclin; neutrophil elastase; protein kinase; monocyte chemotactic proteins 1 and 2 (MCP-1, MCP-2); macrophage migration inhibitory factor (MIF), high mobility group box protein 1 (HMGB-1), and other known factors.

Anti-inflammatory cytokines are also known in the art. Examples of these include IL-4, IL-10, IL-17, IL-13, IL-1a, and TNFα receptor.

It will be recognized that some of the pro-inflammatory cytokines may act as anti-inflammatory cytokines in certain circumstances, and vice-versa. Such cytokines are typically referred to as pleiotropic cytokines.

In some embodiments, stimulation of the splenic arterial nerve may result in: (a) a decrease in the level of an anti-inflammatory cytokine in the plasma or serum by ≤5%, ≤10%, ≤15%, ≤20%, ≤25%, ≤30%, ≤35%, ≤40%, ≤45%, ≤50%, ≤60%, ≤70%, ≤80%, ≤90%, or ≤ 95%; and/or (b) an increase in the level of a pro-inflammatory cytokine in the plasma or serum by ≤5%, ≤10%, ≤15%, ≤20%, ≤25%, ≤30%, ≤35%, ≤40%, ≤45%, ≤50%, ≤60%, ≤70%, ≤80%, ≤90%, ≤95%, ≤100%, ≤150% or ≤200%.

In this context the invention may be useful for increasing an immune response in a subject. For example, increasing an immune response or a pro-inflammatory response may be beneficial in a subject who is immunocompromised and thus in need of increasing pro-inflammatory cytokines for inducing beneficial pro-inflammatory responses. This may be particularly beneficial in immunocompromised subjects who are particularly vulnerable to infections. Examples of immunocompromised subjects in which this embodiment of the invention may be useful include, but are not limited to, subjects undergoing chemotherapy, subjects with HIV or AIDS, subjects taking a course of steroids, and subjects with immunosenescence, for example, subjects with age-associated immunodeficiency. In some embodiments, the invention may be used to increase a pro-inflammatory response in a subject wherein that subject is undergoing or is about to undergo a therapy in which immunocompromisation is an undesired side effect of that therapy. In other embodiments, the invention is useful for inducing a pro-inflammatory response to boost the acquisition of resistance provided by a vaccine. In other words, the neurostimulation device of the invention may be used in a method of vaccination, e.g. to boost the efficacy of a vaccine.

Factors involved in immune responses may be useful measurable parameters in the context of the invention, for example, TGF, PDGF, VEGF, EGF, FGF, I-CAM, nitric oxide.

Chemokines may also be useful measurable parameters in the context of the invention, such as 6cKine and MIP4eta, and chemokine receptors, including CCR7 receptor.

Changes in immune cell population (Langerhans cells, dendritic cells, lymphocytes, monocytes, macrophages), or immune cell surface co-stimulatory molecules (Major Histocompatibility, CD80, CD86, CD28, CD40) may also be useful measurable parameters in the context of the invention. Applying a signal to the nerves according to the invention can cause a reduction in the total counts of circulating or tissue-specific (e.g. joint-specific in the case of rheumatoid arthritis) leukocytes (including monocytes and macrophages, lymphocytes, neutrophils, etc.).

Factors involved in the inflammatory cascade may also be useful measurable parameters in the context of the invention. For example, the signal transduction cascades include factors such as NFκ-B, Egr-1, Smads, toll-like receptors, and MAP kinases.

Methods of assessing these physiological parameters are known in the art. Detection of any of the measurable parameters may be done before, during and/or after modulation of neural activity in the nerve.

For example, a cytokine, chemokine, or a catecholamine (e.g. norepinephrine or epinephrine) may be directly detected, e.g. by ELISA. Alternatively, the presence or amount of a nucleic acid, such as a polyribonucleotide, encoding a polypeptide described herein may serve as a measure of the presence or amount of the polypeptide. Thus, it will be understood that detecting the presence or amount of a polypeptide will include detecting the presence or amount of a polynucleotide encoding the polypeptide.

Quantitative changes of the biological molecules (e.g. cytokines) can be measured in a living body sample such as urine or plasma. Detection of the biological molecules may be performed directly on a sample taken from a subject, or the sample may be treated between being taken from a subject and being analyzed. For example, a blood sample may be treated by adding anti-coagulants (e.g. EDTA), followed by removing cells and cellular debris, leaving plasma containing the relevant molecules (e.g. cytokines) for analysis. Alternatively, a blood sample may be allowed to coagulate, followed by removing cells and various clotting factors, leaving serum containing the relevant molecules (e.g. cytokines) for analysis.

In the embodiments where the signal is applied whilst the subject is asleep, the invention may involve determining the subject's circadian rhythm phase markers, such as the level of cortisol (or its metabolites thereof), the level of melatonin (or its metabolites thereof) or core body temperature. Cortisol or melatonin levels can be measured in the blood (e.g. plasma or serum), saliva or urine. Methods of determining the levels of these markers are known in the art, e.g. by enzyme-linked immunosorbent assay (ELISA) or radioimmunoassay. If measurements of the subject's circadian rhythm phase markers indicate circadian oscillations of inflammatory markers which may beneficially be regulated by application of a signal with a neurostimulation device or system of the invention, then application of the signal at night at a suitable periodicity according to the subject's circadian rhythm may be appropriate.

As used herein, a physiological parameter is not affected by the modulation (e.g. stimulation) of the splenic neural activity if the parameter does not change (in response to nerve modulation) from the normal value or normal range for that value of that parameter exhibited by the subject or subject when no intervention has been performed, i.e. it does not depart from the baseline value for that parameter. Such a physiological parameter may be arterial pressure, heart rate or glucose metabolism. Suitable methods for determining changes in any these physiological parameters would be appreciated by the skilled person.

The skilled person will appreciate that the baseline for any neural activity in a subject need not be a fixed or specific value, but rather can fluctuate within a normal range or may be an average value with associated error and confidence intervals. Suitable methods for determining baseline values are well known to the skilled person.

As described herein, a physiological parameter is determined in a subject when the value for that parameter exhibited by the subject at the time of detection is determined. A detector (e.g. a physiological sensor subsystem, a physiological data processing module, a physiological sensor, etc.) is any element able to make such a determination.

Thus, in certain embodiments, the method according to this aspect of the invention further comprises a step of determining one or more physiological parameters of the subject, wherein the signal is applied only when the determined physiological parameter meets or exceeds a predefined threshold value. In such embodiments wherein more than one physiological parameter of the subject is determined, the signal may be applied when any one of the determined physiological parameters meets or exceeds its threshold value, alternatively only when all of the determined physiological parameters meet or exceed their threshold values. In certain embodiments, the signal is applied by a system of the invention, which in addition to the neurostimulation device comprises at least one detector configured to determine the one or more physiological parameters of the subject.

In certain embodiments, the physiological parameter is an action potential or pattern of action potentials in a nerve of the subject, wherein the action potential or pattern of action potentials is associated with the disease, disorder or condition to be treated.

A predefined threshold value for a physiological parameter is defined elsewhere herein.

A subject of the invention may, in addition to being treated with a neurostimulation device or system according to the invention, receive medicine for their disease, disorder or condition, as discussed elsewhere herein. In the methods of the invention, anticoagulant therapy, e.g. with heparin, may be administered to the subject prior to, following, and/or simultaneously with the application of the neurostimulation device of the invention.

### Suitable Forms of an Electrical Signal

The neurostimulation device according to the invention applies an electrical signal via at least one electrode which is placed in proximity to, i.e. in a signalling relationship with, a splenic arterial nerve when the distal end of the catheter or stent of the neurostimulation device is inserted into a blood vessel, for example a splenic artery. The electrode may be said to be placed in signalling contact with the splenic arterial nerve. As used herein, “signalling contact” is where at least part of the electrical signal applied via the at least one electrode is received at the nerve.

Electrical signals applied according to the invention (in any medical setting described herein) may be non-destructive. As used herein, a “non-destructive signal” is a signal that, when applied, does not irreversibly damage the underlying neural signal conduction ability of the nerve. That is, application of a non-destructive signal maintains the ability of the nerve or fibers thereof, or other nerve tissue to which the signal is applied, to conduct action potentials when application of the signal ceases, even if that conduction is in practice artificially stimulated as a result of application of the non-destructive signal.

Electrical signals applied according to the invention may be a voltage or a current waveform (e.g. constant voltage or a constant current waveform).

The electrical signal may be characterized by one or more electrical signal parameters. The electrical signal parameters include waveform, frequency, and amplitude.

Alternatively or additionally, the electrical signal may be characterized by the pattern of application of the electrical signal to the nerve. The pattern of application refers to the timing of the application of the electrical signal to the nerve. The pattern of application may be continuous application or periodic application.

Continuous application refers to a situation in which the electrical signal is applied to the nerve in a continuous manner. In embodiments where the electrical signal is a series of pulses, the gaps between those pulses (i.e. between the pulse width and the phase duration) do not mean that the signal is not continuously applied.

Periodic application refers to where the electrical signal is applied to the nerve in a repeating pattern (e.g. an on-off pattern).

In the context of the treatment of a disease, disorder or condition associated with inflammation, e.g. an inflammatory disorder, e.g. an autoimmune disorder, the pattern of application of the electrical signal may be continuous application, periodic application and/or episodic application. Episodic application refers to where the electrical signal is applied to the nerve for a discrete number of episodes throughout a day. Each episode may be defined by a set duration or a set number of iterations of the electrical signal. Where the electrical signal is applied periodically and episodically, it means that the signal is applied in a periodic manner for each episode of application. Where the electrical signal is applied continuously and episodically, it means that the signal is applied in a continuous manner for each episode of application.

The inventors have found preferred electrical signal parameters and patterns of signal application for stimulating neural activity in a splenic arterial nerve by applying the signal to the application site for use in accordance with the invention, which parameters and/or patterns lead to increased immunosuppressive effects while reducing possible systemic effects when stimulating neural activity in said nerve. The preferred signal parameters and patterns of application are discussed in detail below.

The inventors have also found improved waveforms of the electrical signal which decrease the pulse height required in order to stimulate neural activity in a human nerve supplying the spleen, whilst reducing the burden on the stimulator. The improved waveforms are discussed in detail below.

### Waveform

Modulation (e.g. stimulation) of a nerve e.g. supplying the spleen can be achieved using electrical signals applied by the neurostimulation device (stent) of the invention.

A pulse train comprises a plurality of sequential pulses, where each pulse may be characterized by pulse width, pulse height and/or interphase delay. Pulse width refers to a width (or time duration) of a primary phase of the waveform. In some cases where a pulse comprises a first phase that is the primary phase and a second phase which is the recovery phase, for example an anodic and/or a cathodic phase, the pulse width refers to a width (or duration) of the first phase. A pulse duration refers to the time duration during which the pulse is applied or delivered for. This may also be referred to as a stimulation time.

Interphase delay refers to the time period from the end of a pulse to the start of the next pulse. Pulse height, which is also referred to as pulse amplitude, refers to the amplitude of current of the pulse, typically measured in amps.

Pulse width and pulse height are preferably constant for all of the pulses in the pulse train. Likewise, interphase delay is preferably constant between all of the pulses in the pulse train.

Through experimental studies, the inventors have found improved waveforms of the electrical signal which decrease the pulse height required in order to stimulate neural activity in a human nerve supplying the spleen, thereby optimizing the biological efficacy and reproducibility of stimulation parameters of the electrical signal for use in humans whilst reducing the burden on the stimulator. Thus, the electrical signal may comprise a pulse train having a pulse width >0.1 ms, optionally ≥0.4 ms, optionally ≥1 ms, optionally >1 ms. Additionally or alternatively, the pulse width may be ≤5 ms, optionally ≤3 ms, optionally ≤ 2 ms. Optionally, the pulse width may be between 0.1 and 5 ms, optionally between 0.4 and 4 ms, optionally between 1 and 3 ms, optionally between 1.5 and 2.5 ms, optionally between 1.75 ms and 2.25 ms, optionally between 1.9 ms and 2.1 ms, optionally 2 ms. Moreover, the pulse train may have an interphase delay of ≤0.3 ms, more optionally ≤0.25 ms. Additionally or alternatively, the interphase delay may be ≥ Oms, ≥0.1 ms, optionally ≥0.2 ms, more optionally 0.2 ms.

With an increased pulse width, a decrease in the pulse height required to stimulate neural activity in a human splenic nerve is observed. The pulse height required to stimulate neural activity in a nerve is also referred to herein as the ‘stimulation threshold’ and the ‘pulse height threshold’.

The inclusion of an interphase delay may reduce the threshold of pulse height required to stimulate neural activity in a human splenic nerve. Therefore, in some examples, the pulse train may have an interphase delay.

Longer interphase delays may produce greater reductions in pulse height threshold. Accordingly, the interphase delay may have a lower limit of ≥0 ms, ≥0.1 ms, optionally ≥0.15 ms, optionally ≥0.19 ms, optionally ≥0.2 ms. At interphase delays greater than 0.3 ms it was found that there is no further reduction in pulse height threshold. Accordingly, the upper limit of interphase delay of the pulse train may be ≤0.3 ms, more optionally ≤0.25 ms. Any combination of the upper and lower limits of interphase delay is possible. Preferred ranges of interphase delay include between 0.1 ms and 0.3 ms, and between 0.2 ms and 0.25 ms. The pulses are optionally square pulses. However, other pulse waveforms such as sawtooth, sinusoidal, triangular, trapezoidal, quasitrapezodial or complex waveforms may also be used with the invention.

The pulses may be biphasic in nature. The term “biphasic” refers to a pulse which applies to the nerve over time both a positive and negative charge (anodic and cathodic phases). For biphasic pulses, the pulse width includes the time duration of a primary phase of the waveform, for example the anodic phase or the cathodic phase. The primary phase may also be referred to herein as the stimulation phase.

The pulses may be charge-balanced. A charge-balanced pulse refers to a pulse which, over the period of the pulse, applies equal amounts (or thereabouts) of positive and negative charge to the nerve. The biphasic pulses are preferably charge-balanced.

The pulses may be symmetric or asymmetric. A symmetric pulse is a pulse where the waveform when applying a positive charge to the nerve is symmetrical to the waveform when applying a negative charge to the nerve. An asymmetric pulse is a pulse where the waveform when applying a positive charge to the nerve is not symmetrical with the waveform when applying a negative charge to the nerve.

If the biphasic pulse is asymmetric, but remains charged balanced, then the areas of the opposing phases must equal. Amplitude (see below) can be reduced, but the pulse width would need to be extended to ensure the area under the curve is matched.

In an exemplary embodiment, the waveform is a pulse train with biphasic, asymmetric, charge balanced square pulses.

### Amplitude

For the purpose of the invention, the amplitude is referred to herein in terms of charge density per phase. Charge density per phase applied to the nerve by the electrical signal is defined as the integral of the current over one phase (e.g. over one phase of the biphasic pulse in the case of a charge-balanced biphasic pulse) over a stimulating electrode surface area. Thus, charge density per phase applied to the nerve by the electrical signal is the charge per phase per unit of surface area of the at least one electrode intravascularly, and also the integral of the current density over one phase of the signal waveform. Put another way, the charge density per phase applied to the nerve by the electrical signal is the charge per phase applied to the nerve by the electrical signal divided by the surface area of the at least one electrode (generally the cathode) intravascularly.

The charge density per phase that is useful for the invention represents the amount of energy required to stimulate neural activity in a nerve supplying the spleen to increase immunosuppressive effects.

The inventors found the current that is useful to stimulate neural activity in a splenic arterial nerve to be between 1 mA and 400 mA, 1 mA and 100 mA; preferably between 5 mA and 50 mA, optionally between 5 mA and 100 mA, preferably between 10 mA and 40 mA, preferably between 20 mA and 30 mA.

The charge density per phase required to input to stimulate neural activity in a human splenic arterial nerve may be ≤4000 μC per cm² per phase, optionally between 20 μC to 3500 μC per cm² per phase, optionally between 50 μC to 3000 μC per cm² per phase, optionally between 200 μC to 2000 μC per cm² per phase, optionally between 300 μC to 1800 μC per cm² per phase, optionally between 400 μC to 1500 μC per cm² per phase, optionally between 500 μC to 1500 μC per cm² per phase.

For example, the charge density per phase applied by the electrical signal may be ≤100 μC per cm² per phase, ≤150 μC per cm² per phase, ≤200 μC per cm² per phase, ≤250 ρC per cm² per phase, ≤300 μC per cm² per phase, ≤400 μC per cm² per phase, ≤500 μC per cm² per phase, ≤750 μC per cm² per phase, ≤1000 μC per cm² per phase, ≤1250 μC per cm² per phase, or ≤1500 μC per cm² per phase. Additionally or alternatively, the charge density per phase applied by the electrical signal may be ≥50 μC per cm² per phase, ≥100 μC per cm² per phase, ≥150 μC per cm² per phase, ≥200 μC per cm² per phase, ≥250 μC per cm² per phase, ≥300 μC per cm² per phase, ≥400 μC per cm² per phase, ≥500 ρC per cm² per phase, ≥750 μC per cm² per phase, ≥1000 μC per cm² per phase, or ≥1250 μC per cm².

The total charge applied to the nerve by the electrical signal in any given time period is a result of the charge density per phase of the signal, in addition to the frequency of the signal, the pattern of application of the signal and the surface area of at least one electrode intravascularly. The frequency of the signal, the pattern of application of the signal and the surface area of at least one electrode intravascularly are discussed further herein.

It will be appreciated by the skilled person that the amplitude of an applied electrical signal necessary to achieve the intended stimulation of the neural activity will depend upon the positioning of the electrode and the associated electrophysiological characteristics (e.g. impedance). It is within the ability of the skilled person to determine the appropriate current amplitude for achieving the intended modulation of the neural activity in a given subject.

It would be of course understood in the art that the electrical signal applied to the nerve would be within clinical safety margins (e.g. suitable for maintaining nerve signaling function, suitable for maintaining nerve integrity, and suitable for maintaining the safety of the subject). The electrical parameters within the clinical safety margin would typically be determined by pre-clinical studies.

The table below demonstrates example electrical signal parameters for each corresponding recruitment level of human splenic nerve using computational models. These are example values only, where a different current amplitude or pulse width may be used depending on the electrode surface area or electrode configuration of a device to achieve a corresponding charge density. In the examples given below, the electrode area is assumed to be 0.067 cm². A range around the example values provided may also be used.

| | | Recruitment (10%) | | | | Recruitment (50%) | | | | Recruitment (100%) | |
|---|---|---|---|---|---|---|---|---|---|---|---|
| Pulse | | Stimulation | | Charge | | Stimulation | | Charge | | Stimulation | Charge |
| width | | amplitude | | densities | | Amplitude | | densities | | Amplitude | densities |
| 200 | μs | ~84.2 | mA | ~250 | μC/cm² | ~139.7 | mA | ~417 | μC/cm² | ~385.2 mA | ~1149 μC/cm² |
| 400 | μs | ~43 | mA | ~250 | μC/cm² | ~75.25 | mA | ~449 | μC/cm² | ~209.6 mA | ~1251 μC/cm² |
| 600 | μs | ~28.67 | mA | ~250 | μC/cm² | ~55.55 | mA | ~497 | μC/cm² | ~152.3 mA | ~1363 μC/cm² |
| 800 | μs | ~21.5 | mA | ~250 | μC/cm² | ~44.79 | mA | ~534 | μC/cm² | ~130.8 mA | ~1561 μC/cm² |
| 1000 | μs | ~17.92 | mA | ~267.3 | μC/cm² | ~39.41 | mA | ~590 | μC/cm² | ~112.9 mA | ~1685 μC/cm² |
| 2000 | μs | ~16.12 | mA | ~481 | μC/cm² | ~37.62 | mA | ~1123 | μC/cm² | ~107.5 mA | ~3208 μC/cm² |

### Periodic Application

Periodic application refers to where the electrical signal is applied to the nerve in a repeating pattern. The preferred repeating pattern is an on-off pattern, where the signal is applied in a sequence of pulse trains for a first duration, referred to herein as an ‘on’ duration, then stopped for a second duration, referred to herein as an ‘off’ duration, then applied again for the first duration, then stopped again for the second duration, etc.

The periodic on-off pattern may have an on duration of between 0.1 and 10 s and an off duration of between 0.5 and 30 s. For example, the on duration may be ≤0.2 s, ≤0.5 s, ≤1 s, ≤2 s, ≤5 s, or ≤10 s. Alternatively or additionally, the on duration may be ≥0.1 s, ≥0.2 s, ≥0.5 s, ≥1 s, ≥2 s, or ≥5 s. Any combination of the upper and lower limits above for the on duration is also possible. For example, the off duration may be ≤1 s, ≤3 s, ≤5 s, ≤10 s, ≤15 s, ≤20 s, ≤25 s, or ≤30 s. Alternatively or additionally, the off duration may be ≥0.5 s, ≥1 s, ≥2 s, ≥5 s, ≥10 s, ≥15 s, ≥20 s, or ≤25 s. Any combination of the upper and lower limits above for the off duration is also possible.

In an exemplary embodiment, the periodic on-off pattern has an on duration of 0.5 s on, and an off duration of 4.5 sec off.

Periodic application may also be referred to as a duty cycled application. A duty cycle represents the percentage of time that the signal is applied to the nerve for a cycle of the periodic pattern. For example, a duty cycle of 20% may represent a periodic pattern having an on duration of 2 s, and an off duration of 10 s. Alternatively, a duty cycle of 20% may represent a periodic pattern having a on duration of 1 s, and an off duration of 5 s.

Duty cycles suitable for the present invention are between 0.1% and 100%. For example, the duty cycle may be 10%.

### Episodic Application

Episodic application refers to where the electrical signal is applied to the nerve for a discrete number of episodes throughout a day. The electrical signal according to the invention may be applied for up to a maximum of twenty six episodes per day. For example, the number of episodes of signal application per day may be one, two, three, four, five, six, seven, eight, nine, ten, eleven, twelve or another number up to twenty six.

The electrical signal may be applied episodically every 2 to 3 hours. For example, the electrical signal may be applied episodically once every 2 hours, 2 hour 15 min, 2 hour 30 min, 2 hour 45 min, or 3 hours.

Each episode may be defined by a set duration or a set number of iterations of the electrical signal. In some embodiments, each episode comprises applying to the nerve between 10 and 2400 pulses of the electrical signal, optionally between 100 and 2400 pulses of the electrical signal, further optionally between 50 and 2400 pulses, e.g. between 200 and 1200 pulses of the electrical signal, between 400 and 600 pulses of the electrical signal, etc. For example, each episode may comprise applying ≤10, ≤50, ≤60, ≤100, ≤400, ≤600, ≤800, ≤1200, ≤1600, ≤2000, or ≤2400 pulses of the electrical signal. In another example, each episode may comprise applying ≤200, ≤400, ≤600, ≤800, ≤1000, or ≤1200 pulses of the electrical signal. In a further example, each episode may comprise applying ≤400, ≤425, ≤ 450, ≤475, ≤500, ≤525, ≤550, ≤575, or ≤600 pulses of the electrical signal.

In other embodiments, each episode comprises between 20 and 40 iterations of the periodic pattern. For example, each episode comprises applying 20, 25, 30, 35, or 40 iterations of the periodic pattern, or any number therebetween. The higher the frequency, the lower the number of iterations.

As mentioned previously, in some embodiments, the episodes may be based on the subject's sleep-wake cycle, in particular the episodes may be whilst the subject is asleep. In some such embodiments, the episodes may be applied between 10 μm and 6 am. This may also be influenced by the surgery times. The sleep-wake cycle may be measured via known methods by detecting the subject's circadian rhythm phase markers (e.g. cortisol level, melatonin level or core body temperature), and/or a detector for detecting the subject's movements.

### Frequency

Frequency is defined as the reciprocal of the phase duration of the electrical waveform (i.e. 1/phase), or put another way the interpulse timing (pulse to pulse timing).

The inventors have found preferred frequencies for stimulating a splenic arterial nerve when using the device of the invention. In particular, the inventors have found preferred frequencies for embodiments where the electrical signal is applied periodically and for embodiments where the electrical signal is applied continuously.

In embodiments where the electrical signal is applied periodically, the electrical signal has a frequency of ≤300 Hz, preferably ≤50 Hz, more preferably ≤10 Hz. For example, the frequency of the electrical signal may be ≤50 Hz, ≤100 Hz, ≤150 Hz, ≤200 Hz, ≤250 Hz or ≤300 Hz. In other examples, the frequency of the electrical signal may be ≤10 Hz, ≤15 Hz, ≤20 Hz, ≤25 Hz, ≤30 Hz, ≤35 Hz, ≤40 Hz, ≤45 Hz, or ≤50 Hz. In further examples, the frequency may be ≤1 Hz, ≤2 Hz, ≤5 Hz, or ≤10 Hz. Additionally or alternatively, the frequency of the electrical signal may be ≥10 Hz, ≥15 Hz, ≥20 Hz, ≥25 Hz, ≥30 Hz, ≥35 Hz ≥40 Hz, ≥45 Hz, or ≥50 Hz. In other examples, the frequency of the electrical signal may be ≥0.1 Hz, ≥0.2 Hz, ≥0.5 Hz, ≥1 Hz, ≥2 Hz, or ≥5 Hz. Any combination of the upper and lower limits above is also possible.

In embodiments where the electrical signal is applied continuously, the electrical signal has a frequency of ≤50 Hz, preferably ≤10 Hz, more preferably ≤2 Hz, even more preferably ≤ 1 Hz. For example, the frequency may be ≤1 Hz, ≤2 Hz, ≤5 Hz, or ≤10 Hz. In other examples the frequency may be ≤0.1 Hz, ≤0.2 Hz, ≤0.3 Hz, ≤0.4 Hz≤0.5 Hz, ≤0.6 Hz≤ 0.7 Hz, ≤0.8 Hz, or ≤0.9 Hz. Additionally or alternatively, the frequency of the electrical signal may be ≥0.1 Hz, ≥0.2 Hz, ≥0.5 Hz, ≥1 Hz, ≥2 Hz, or ≥5 Hz. Any combination of the upper and lower limits above is also possible.

Where the signal waveform comprises a pulse train, the pulses are applied to the nerve at intervals according to the above-mentioned frequencies. For example, a frequency of 50 Hz results in 50 pulses being applied to the nerve per second.

### General Definitions

The term “comprising” encompasses “including” as well as “consisting” e.g. a composition “comprising” X may consist exclusively of X or may include something additional e.g. X+Y. The term “around” or “about” in relation to a numerical value is optional and means, for example, x±10%. Unless otherwise indicated each embodiment as described herein may be combined with another embodiment as described herein.

Any range or device value given herein may be extended or altered without losing the effect sought, as will be apparent to the skilled person.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method blocks or elements identified, but that such blocks or elements do not comprise an exclusive list and a method or apparatus may contain additional blocks or elements.

The steps of the methods described herein may be carried out in any suitable order, or simultaneously where appropriate. Additionally, individual blocks may be deleted from any of the methods without departing from the spirit and scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art.

Although various embodiments have been described above with a certain degree of particularity, or with reference to one or more individual embodiments, those skilled in the art could make numerous alterations to the disclosed embodiments without departing from the scope of this invention.

1. A stent for intravascular neural stimulation, the stent comprising:
a scaffold extending in a longitudinal direction and having an outer perimeter positioned, in use, to at least partially contact the vessel wall; and
a pulse generator configured to generate electrical signals for delivery to a nerve for intravascular neural stimulation;
wherein the scaffold has mounted thereon a first set of one or more electrodes electrically coupled to the pulse generator;
wherein the stent further comprises a second set of one or more electrodes electrically coupled to the pulse generator, said second set of electrodes being unconnected to the scaffold; and
further wherein at least a part of the first and/or second set of electrodes are configured to be aligned with at least a part of the outer perimeter of the scaffold such that the set of electrodes are in signalling contact with the vessel wall when the stent is in situ.

2. The stent of claim 1, wherein the scaffold comprises a proximal end and a distal end; and wherein the first or second set of one or more electrodes are configured such that they extend distally of the distal end of the scaffold.

3. The stent of claim 1, wherein the second set of one or more electrodes are mounted to the pulse generator.

4. The stent of claim 2, wherein the pulse generator is attached to the scaffold and extends beyond the distal end of the scaffold; and wherein the one or more electrodes are mounted to the pulse generator such that the one or more electrodes are positioned distally of the distal end of the scaffold.

5. The stent of claim 1, wherein the pulse generator is attached to an inner perimeter of the scaffold.

6. The stent of claim 1, wherein the pulse generator is attached to an outer perimeter of the scaffold.

7. The stent of claim 6, wherein the scaffold comprises one or more platforms, each platform extending around at least part of the circumference of the scaffold; and wherein the pulse generator is housed in one of the one or more platforms, optionally wherein the stent comprises a plurality of platforms and a corresponding plurality of pulse generators, each housed in a respective platform.

8. The stent of claim 7, wherein each platform comprises a surface that extends radially inwardly to form a trough; and wherein the or each pulse generator is housed in the trough of the respective one of the one or more platforms.

9. The stent of claim 8, wherein the or each pulse generator and the trough in which it is housed are configured such that the radially outermost part of the pulse generator is aligned with at least a part of the outer perimeter of the scaffold, and wherein the one or more electrodes are mounted on the radially outermost part of the or each pulse generator such that, in use, the one or more electrodes at least partially contact the vessel wall.

10. The stent of claim 9, wherein the pulse generator extends at least partially along the length of the scaffold, optionally fully along the length of the scaffold.

11. The stent of claim 10, wherein the pulse generator extends distally of a distal end of the platform; and wherein the one or more electrodes are mounted to the pulse generator such that the one or more electrodes are positioned distally of the distal end of the scaffold.

12. The stent of claim 3, wherein the one or more electrodes of the pulse generator is comprised of a conductive cap mounted at the distal end of the pulse generator.

13. The stent of claim 12, wherein the cap comprises a rim that at least partially surrounds the distal end of the pulse generator and which is coplanar with at least part of the vessel wall when the stent is in situ.

14. The stent of claim 1, wherein at least a portion of either or both of the scaffold and the pulse generator comprises or is formed from an insulating material such that the one or more electrodes mounted to the pulse generator are electrically isolated from the one or more electrodes mounted to the scaffold.

15. The stent of claim 14, wherein the pulse generator comprises a housing to which the one or more electrodes are mounted, and wherein the housing is formed from an insulating material selected from the group consisting of: a ceramic material and an epoxy.

16-17. (canceled)

18. The stent of claim 1, wherein the first set of one or more electrodes have a first polarity, in use, and wherein the second set of one or more electrodes have a second polarity, in use, wherein the first polarity is different from the second polarity.

19. The stent of claim 1, wherein the first set of one or more electrodes are stimulating electrodes and the second set of one or more electrodes are return electrodes.

20. The stent of claim 1, wherein the second set of one or more electrodes are stimulating electrodes and the first set of one or more electrodes are return electrodes.

21. The stent of claim 1, wherein the scaffold is substantially tubular and configured to conform to the walls of the vessel when the stent is in situ.

22. A method of implanting a stent into a vessel at an intravascular location using a deployment catheter, wherein the stent comprises:
a pulse generator;
a scaffold configured to be withdrawn into the deployment catheter; and
a distal set of one or more electrodes electrically coupled to the pulse generator, said distal set of electrodes being unconnected to the scaffold;
the method comprising:
positioning a distal end of the deployment catheter at the intravascular location;
advancing the stent within the deployment catheter until it approaches the distal end of the deployment catheter;
further advancing the stent to expose the one or more electrodes of the distal set of one or more electrodes outside of, preferably beyond the deployment catheter;
providing an electrical stimulation at the intravascular location via the exposed electrodes; and
withdrawing the stent within the deployment catheter.

23-29. (canceled)
